(19) United States
(12) Patent Application Publication
(10) Pub. No.: US 2023/0165559 A1
(43) Pub. Date: Jun. 1, 2023

(54) VESSEL MEASUREMENT DEVICE AND PROCESS

(71) Applicant: STELECT PTY. LTD., Richmond, Victoria (AU)

(72) Inventors: Elise SUTHERLAND, Victoria (AU); Anurupa SHAW, Grenoble (FR); Fay GIBSON, Victoria (AU); Marc STRINGER, Victoria (AU)

(21) Appl. No.: 17/995,404

(22) PCT Filed: Apr. 2, 2021
(86) PCT No.: PCT/IB2021/000240

§ 371 Date: Oct. 3, 2022

Related U.S. Application Data
(60) Provisional application No. 63/005,184, filed on Apr. 3, 2020.
(60) Provisional application No. 63/009,413, filed on Apr. 13, 2020.

(51) Int. Cl. A61B 8/08; A61B 8/00

(52) U.S. Cl. CPC A61B 8/0891; A61B 8/445; A61B 8/4494; A61B 8/463; A61B 2562/043

(57) ABSTRACT

Imaging catheter devices that include one or more sensors, such as one or more ultrasound sensors, for measuring a lumen of a vessel. The catheter devices may include an inflatable balloon. In some cases, the ultrasound sensors include piezoelectric transducers having high resolution and tissue penetration capability while having a small diameter for incorporation into the catheter device. In some cases, the catheter devices include a flexible distal tip for navigating through the vessel.

### CROSS REFERENCE TO RELATED APPLICATIONS

This application claim priority to U.S. Provisional Patent Application No. 63/005,184, filed on Apr. 3, 2020, entitled “VESSEL MEASUREMENT DEVICE AND PROCESS,” and U.S. Provisional Patent Application No. 63/009,413, filed on Apr. 13, 2020, entitled “ULTRASOUND TRANSDUCERS,” each of which is incorporated by reference herein in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

This technology relates to measuring of vessels within humans or animals using intravascular devices. An application of an example is for imaging a coronary vessel to provided data to assist clinicians and/or surgeons in making choices for coronary stents for subsequent implant.

### BACKGROUND

Coronary and other vascular stenting procedures are becoming routine surgeries. However, the methods commonly used for selection of stent size are often subject to “educated guesses” on the part of the clinicians and/or surgeons, which can lead to errors and lead to poor health outcomes for the patients.

Products available to clinicians and/or surgeons to measure internal vascular geometry and assist with stent selection include including IVUS (intravascular ultrasound) and OCT (optical coherence tomography) however their uptake was minimal around the world. IVUS is a catheter mounted ultrasound imaging device that is used to image inside of a coronary vessel. OCT uses a catheter device to deliver and collect near infrared light to create cross-sectional images of artery lumen. A problem with these types of catheter devices is that they are specific to imaging and so must be inserted for the imaging and then removed before the procedure (for example insertion of a stent) can be performed. Many clinicians find these devices time consuming and difficult to use. Further, the devices currently available commercially do not integrate well with other catheterization laboratory (Cath Lab) equipment and procedures. There is also significant expense associated with the equipment and consumable devices for such imaging technologies.

Having to separately insert and remove the imaging catheter can significantly lengthen procedure time, typically adding 15 minutes to the time required to complete the procedure. Typical time for a procedure such as inserting a cardiac stent is around 50 minutes to an hour, thus procedure time may be increased by around 25% if an imaging catheter is used. Increased procedure time may increase the risk of medical complications. Additional procedure time and the additional equipment can significantly increase the overall expense of the surgery for each patient. Further hospital surgical facilities and Cath Labs are fixed resources, so increased procedure time may reduce the number of patients able to be treated and thus restrict patient access to life saving treatment.

There is need for improved integration of vascular imagine technology and catheterization procedures.

### SUMMARY OF THE DISCLOSURE

According to one aspect there is provided an imaging balloon catheter device comprising: an inflatable balloon element; an elongate tubular body having a distal end and a proximal end, the distal end for insertion into a vessel and the proximal end configured to remain external to the vessel accessible to a clinician and connectable to external components, the distal end being configured to support the inflatable balloon element and enable inflation and deflation of the balloon element; and an imagining sensor assembly mounted proximate the inflatable balloon element at the distal end of the tubular body, the imaging sensor assembly being in data communication with an image processing system configured to process data output from the imaging sensor assembly and render for display.

In some examples the imaging sensor assembly is configured to enable 360 degree imaging of the vessel interior.

In some examples the imaging sensor assembly is a sensor array comprising one or more sensors. In one example the one or more sensors of the array are arranged concentrically around the tubular body. In another example the one or more sensors are arranged in a linear array.

In some examples of the imaging balloon catheter the tubular body supports one or more electrical conductors to provide power and data connection to the sensor assembly. In one example the electrical conductors are wires arranged to connect each of the sensors in the array to a bus extending longitudinally along an internal lumen wall of the catheter body.

In some examples of the imaging balloon catheter the imaging sensor assembly is an ultrasound sensor assembly.

In other examples of the imaging balloon catheter the imaging sensor array is configured for any one or more of electromagnetic induction tomography, electrical capacitance tomography, electrical resistance tomography, electrical induction tomography, near infrared spectroscopy, optical coherence tomography, photography or videography.

Another aspect provides an imaging balloon catheter system comprising an imaging balloon catheter as described above and a processor configured to analyze and display image data.

In some examples of the imaging balloon catheter system the processor is configured to monitor movement of the catheter as the catheter is extracted, and wherein movement data is input to analysis of sampled image data.

In some examples the imaging balloon catheter system further comprises a mechanism for controlling extraction of the catheter, whereby the catheter can be extracted at a pre-set pull back rate.

In some examples monitoring movement of the catheter is based on marker detection.

In some examples the imaging balloon catheter system further comprises one or more motion sensors mounted on the catheter body to enable monitoring of movement of the catheter.

Another aspect provides a method of imaging within a vessel during a catheterization procedure comprising the steps of: monitoring position within a vessel of an imaging sensor array mounted on a balloon catheter; receiving an input triggering start of imaging; in response receiving the trigger: monitoring movement of the catheter to provide movement data characterizing movement of the catheter; sampling image data using the imaging array; processing the sampled imaged data using movement data to render images of the vessel; and display the image data.

In some examples the method further comprises the step of determining a treatment area length based on the sampled image data.

In some examples the treatment area length is determined based on identification of a start and end of a vessel wall abnormality based on the image data and calculation of a distance between the start and end.

The present invention also provides a combined imaging balloon catheter, comprising: a catheter having a proximal end and a distal end with a flexible tip; an inflatable balloon assembly on the catheter proximal to the flexible tip; and an imaging assembly in the flexible tip distal to the inflatable balloon assembly; wherein the flexible tip has a length and flexibility sufficient to house the imaging assembly for imaging a portion of a blood vessel or lumen to be dilated or stented, but insufficient to puncture a wall of the blood vessel or lumen.

The present invention further provides a method of sizing a stent for a blood vessel or lumen, the method comprising: imaging a portion of the blood vessel or lumen to be stented using the imaging assembly of the above combined imaging balloon catheter without the inflatable balloon assembly being passed wholly through the portion, and then being pulled wholly back through the portion of the blood vessel or lumen; determining the stent size based at least in part on the imaging of the portion of the blood vessel or lumen.

Any of the catheters described herein may include piezoelectric micromachined ultrasonic transducer (PMUT) devices arrangements that can operate at high frequencies and at high penetration depths for a given applied voltage, to provide high resolution ultrasound images.

The PMUT devices can include a number of piezoelectric stacks, arranged as a cell (also referred to herein as a “PMUT sensor” or “PMUT”), with each cell including a multilayer stack extending proud of a base layer over a cavity in a substrate. The multilayer stack may include a plurality of piezoelectric layers, each flanked by electrode layers, and at least one base layer to add rigidity to the membrane during vibration. The thicknesses and/or materials of the piezoelectric layer(s) and/or base layer can be chosen to achieve a desired performance of the PMUT device. In some examples, the piezoelectric layers each have a height ranging from 0.25 micrometers to 3 micrometers. In some examples, the base layer has a thickness of at least 500 nanometers. In some examples, the one or more piezoelectric layers includes a lead-free material, such as zinc oxide and/or aluminum nitride.

Thus, the multilayer stack may include two or more piezoelectric layers, which may increase the total displacement of the multilayer stack membrane. As described herein, doubling of the piezoelectric layers may increase total displacement with unit driving voltage compared to a single stack of the same thickness. In cases where the multilayer stack includes two or more piezoelectric layers, the two or more piezoelectric layers may be arranged to have an alternating polarity to provide a uniform electric field inside the stack and for ease of connection, as adjacent piezoelectric layers may be separated by a single electrode. Alternatively, in some examples the piezoelectric layers may be polarized in the same direction, and may be sandwiched between separate electrode layers.

For example, described herein are imaging balloon catheter devices that may include: an inflatable balloon element; an elongate tubular body having a distal end and a proximal end, the distal end for insertion into a vessel and the proximal end configured to remain external to the vessel accessible to a clinician and connectable to external components, the distal end being configured to support the inflatable balloon element and enable inflation and deflation of the inflatable balloon element; and an imaging sensor assembly on the elongate tubular body at a distal location relative to the inflatable balloon element, the imaging sensor assembly including an array of piezoelectric micromachined ultrasound transducer (PMUT) sensors arranged in a ring or helix around the elongate tubular body, the imaging sensor assembly being in data communication with an image processing system configured to process data output from the imaging sensor assembly and render for display.

The imaging sensor assembly may be configured to enable 360 degree imaging of the vessel interior. In some examples the PMUT sensors described herein are particularly compact and allow a number of these PMUT sensors (e.g., 3 or more, 4 or more, 5 or more, 6 or more, 7 or more, 8 or more, 9 or more, 10 or more, 11 or more, 12 or more, 15 or more, 20 or more, 25 or more, 30 or more, 35 or more, 40 or more, 45 or more, 50 or more, 55 or more, 60 or more, 65 or more or more, etc.) to be positioned around even the narrow catheter outer surface, permitting the sensors to be compactly arranged, for example as a ring, a helix, or a line. For example, in some examples, more than 60 sensors (e.g., 64 sensors) are arranged around the periphery of the device. The compact nature of these sensors may also allow them to continue to be used to image without substantially reducing the flexibility and/or maneuverability of the catheters described herein (with or without balloons). For example, each PMUT sensor may has a diameter that is 50 μm or less (e.g., 45 μm or less, 40 μm or less, 35 μm or less, 30 μm or less, 25 μm or less, 20 μm or less or less, etc.).

The elongate tubular body may support one or more electrical conductors to provide power and data connection to the imaging sensor assembly. For example, an electrical conductor may include wires connecting each of the PMUT sensors in the array to a bus extending longitudinally along an internal lumen wall of the elongate tubular body.

The imaging sensor assembly may be further configured for any one or more of electromagnetic induction tomography, electrical capacitance tomography, electrical resistance tomography, electrical induction tomography, near infrared spectroscopy, optical coherence tomography, photography or videography. Any of these devices may include a processor configured to analyze and display image data, for example, a processor configured to monitor movement of the imaging balloon catheter device as the imaging balloon catheter device is extracted, and wherein movement data is input to analysis of sampled image data.

Any of the apparatuses (e.g., devices) described herein may include a mechanism for controlling extraction of the imaging balloon catheter device, whereby the imaging balloon catheter device can be extracted at a pre-set pull back rate. Monitoring movement of the imaging balloon catheter device may be based on marker detection via an angiogram feed or via markers on the imaging balloon catheter device external to the vessel and visually accessible to a clinician. Any of these apparatuses (e.g., devices) may include one or more motion sensors mounted on the elongate tubular body to enable monitoring of movement of the imaging balloon catheter device. The one or more motion sensors may comprise one or more of an accelerometer, a gyroscope, a Global Positioning System (GPS) sensor, a velocity sensor, a position sensor, and an optical sensor.

An outer diameter of the imaging balloon catheter device may be, e.g., 3 French or less.

As will be described in greater detail herein, each of the PMUT sensors of the array may include a multilayered stack including a plurality of piezoelectric layers arranged between electrode layers, wherein the multilayered stack is arranged over a cavity of a substrate. For example, each PMUT sensor of the array may comprise a plurality of concentric multilayered stacks extending proud of a base layer, the plurality of concentric multilayered stacks and base layer arranged over a cavity, wherein the concentric multilayered stacks are separated by a space, further wherein each of the concentric multilayered stacks includes a plurality of piezoelectric layers, and wherein each piezoelectric layer is arranged between electrode layers. The base layer may have a thickness of at least 500 nanometers. In any of these apparatuses (e.g., devices), the piezoelectric layers may alternate in polarity along a direction of a height of the stack. Each of the stacks may include between two and eight piezoelectric layers. For example, the piezoelectric layers may each have a thickness ranging from 0.25 micrometers to 3 micrometers. The imaging sensor assembly may have a working frequency between 70 MHz and 80 MHz and has a penetration depth of at least 0.6 cm. In some examples the imaging sensor assembly may have a working frequency between 35 MHz and 45 MHz and has a penetration depth of at least 1 cm. The imaging sensor assembly may have a working frequency between 10 MHz and 20 MHz and has a penetration depth of at least 4 cm.

For example, an imaging balloon catheter device may include: an inflatable balloon element; an elongate tubular body having a distal end and a proximal end, the distal end for insertion into a vessel and the proximal end configured to remain external to the vessel accessible to a clinician and connectable to external components, the distal end being configured to support the inflatable balloon element and enable inflation and deflation of the inflatable balloon element; and an imaging sensor assembly on the elongate tubular body at a distal location relative to the inflatable balloon element, the imaging sensor assembly including an array of piezoelectric micromachined ultrasound transducer (PMUT) sensors on the elongate tubular body, wherein each of the PMUT sensors of the array includes a multilayered stack including a plurality of piezoelectric layers arranged between electrode layers, wherein the multilayered stack is arranged over a cavity of a substrate.

Also described herein are methods, including methods of imaging within a vessel during a catheterization procedure, the method comprising: positioning a balloon catheter within a vessel, the balloon catheter including an imaging sensor assembly on an elongate tubular body, the imaging sensor assembly distally located relative to an inflatable balloon element, the imaging sensor assembly including piezoelectric micromachined ultrasound transducer (PMUT) sensors arranged around the elongate tubular body in a ring or helix; receiving an input triggering start of imaging; and in response to receiving the input: monitoring movement of the balloon catheter to provide movement data characterizing movement of the balloon catheter; sampling image data using the imaging sensor assembly; processing the sampled image data using the movement data to render images of the vessel; and display the sampled image data.

Any of these methods may include determining a treatment area length based on the sampled image data. The treatment area length may be determined based on identification of a start and end of a vessel wall abnormality based on the sampled image data and calculation of a distance between the start and end. Any of these methods may include determining a location of the balloon catheter using a radio opaque marker.

Any of these methods may include rotating the balloon catheter during imaging.

Sampling image data using the imaging sensor assembly may comprise applying a voltage between a plurality of electrode layers in each PMUT sensor, wherein each PMUT comprises a plurality of concentric multilayered stacks, each multilayered stack extending proud of a base layer over a cavity, further wherein each of the concentric multilayered stacks may include a plurality of piezoelectric layers, and wherein each piezoelectric layer is arranged between two electrode layers of the plurality of electrode layers; and inducing, from the applied voltage, a displacement that is a proportional to the applied voltage, a piezoelectric coefficient of a material forming the piezoelectric layers, and the number of piezoelectric layers. Inducing the displacement may comprise inducing a displacement at a frequency of between about 70 MHz and 80 MHz, wherein a penetration depth of an ultrasound signal emitted by the displacement is at least 0.6 cm. In some examples, inducing the displacement comprises inducing a displacement at a frequency of between about 35 MHz and 45 MHz, wherein a penetration depth of an ultrasound signal emitted by the displacement is at least 1 cm. In some examples, inducing the displacement comprises inducing a displacement at a frequency of between about 10 MHz and 20 MHz, wherein a penetration depth of an ultrasound signal emitted by the displacement is at least 4 cm.

For example, a method of imaging within a vessel during a catheterization procedure may include: positioning a balloon catheter within a vessel, the balloon catheter including an imaging sensor assembly on an elongate tubular body, the imaging sensor assembly distally located relative to an inflatable balloon element, the imaging sensor assembly including piezoelectric micromachined ultrasound transducer (PMUT) sensors; receiving an input triggering start of imaging; and in response to receiving the input: monitoring movement of the balloon catheter to provide movement data characterizing movement of the balloon catheter; sampling image data using the imaging sensor assembly by applying a voltage between a plurality of electrode layers in each PMUT sensor, wherein each PMUT comprises a plurality of concentric multilayered stacks, each multilayered stack extending proud of a base layer over a cavity, further wherein each of the concentric multilayered stacks may include a plurality of piezoelectric layers, and wherein each piezoelectric layer is arranged between two electrode layers of the plurality of electrode layers; and inducing, from the applied voltage, a displacement that is a proportional to the applied voltage, a piezoelectric coefficient of a material forming the piezoelectric layers, and the number of piezoelectric layers; processing the sampled image data using the movement data to render images of the vessel; and display the sampled image data.

Also described herein are combined imaging balloon catheter devices comprising: a catheter having a distal end with a flexible tip having an outer diameter of 3 French or less; an inflatable balloon assembly on the catheter proximal to the flexible tip; and an imaging assembly in the flexible tip distal to the inflatable balloon assembly, the imaging assembly including an array of 4 or more piezoelectric micromachined ultrasound transducer (PMUT) sensors arranged in a ring or helix; wherein the flexible tip has a length and flexibility sufficient to house the imaging assembly for imaging a portion of a blood vessel or lumen to be dilated or stented without puncturing a wall of the blood vessel or lumen. The inflatable balloon assembly may be a compliant or non-compliant balloon.

The flexible tip may narrow distally away from one or both of the inflatable balloon assembly and the imaging assembly. As mentioned above, each PMUT sensor of the array may comprise a plurality of concentric multilayered stacks extending proud of a base layer, the plurality of concentric multilayered stacks and base layer arranged over a cavity, wherein the concentric multilayered stacks are separated by a space, further wherein each of the concentric multilayered stacks includes a plurality of piezoelectric layers, and wherein each piezoelectric layer is arranged between electrode layers. The piezoelectric layers may each have a thickness ranging from 0.25 micrometers to 3 micrometers. The array of PMUT sensors may have a working frequency between 70 MHz and 80 MHz and has a penetration depth of at least 0.6 cm. In some examples the array of PMUT sensors has a working frequency between 35 MHz and 45 MHz and has a penetration depth of at least 1 cm. In some examples the array of PMUT sensors has a working frequency between 10 MHz and 20 MHz and has a penetration depth of at least 4 cm.

For example a combined imaging balloon catheter device may include: a catheter having a distal end with a flexible tip having an outer diameter of 3 French or less; an inflatable balloon assembly on the catheter proximal to the flexible tip; and an imaging assembly in the flexible tip distal to the inflatable balloon assembly, the imaging assembly including an array of piezoelectric micromachined ultrasound transducer (PMUT) sensors, wherein each PMUT sensor of the array comprises a plurality of concentric multilayered stacks extending proud of a base layer, the plurality of concentric multilayered stacks and base layer arranged over a cavity, wherein the concentric multilayered stacks are separated by a space, further wherein each of the concentric multilayered stacks includes a plurality of piezoelectric layers, and wherein each piezoelectric layer is arranged between electrode layers; wherein the flexible tip has a length and flexibility sufficient to house the imaging assembly for imaging a portion of a blood vessel or lumen to be dilated or stented without puncturing a wall of the blood vessel or lumen.

Also described herein are methods of sizing a stent for a blood vessel or lumen including the steps of: positioning a combined imaging balloon catheter within the blood vessel or lumen, the combined imaging balloon catheter including: a catheter having a distal end with a flexible tip having an outer diameter of 3 French or less; an inflatable balloon assembly on the catheter proximal to the flexible tip; and an imaging assembly in the flexible tip distal to the inflatable balloon assembly, the imaging assembly including an array of 4 or more piezoelectric micromachined ultrasound transducer (PMUT) sensors arranged in a ring or helix; imaging a portion of the blood vessel or lumen to be stented using the imaging assembly of the combined imaging balloon catheter without the inflatable balloon assembly being passed wholly through the portion of the blood vessel or wholly pulled back through the portion of the blood vessel or lumen; and determining a stent size based at least in part on the imaging of the portion of the blood vessel or lumen.

Determining the stent size may include determining a treatment area length based on collected image data. For example, the treatment area length may be determined based on identification of a start and end of a vessel wall abnormality based on the collected image data and a calculation of a distance between the start and end. Any of these methods may include measuring longitudinal and rotational movement of the combined imaging balloon catheter within the blood vessel or lumen. The inflatable balloon assembly may comprise a compliant or non-compliant balloon, as mentioned above, and the flexible tip may narrow distally away from one or both of the inflatable balloon assembly and the imaging assembly.

In any of these examples imaging the portion of the blood vessel or lumen to be stented may include applying a voltage between a plurality of electrode layers in each PMUT sensor of the array of PMUT sensors, wherein each PMUT sensor comprises a plurality of concentric multilayered stacks, each multilayered stack extending proud of a base layer over a cavity, further wherein each of the concentric multilayered stacks may include a plurality of piezoelectric layers, and wherein each piezoelectric layer is arranged between two electrode layers of the plurality of electrode layers; and inducing, from the applied voltage, a displacement that is a proportional to the applied voltage, a piezoelectric coefficient of a material forming the piezoelectric layers, and the number of piezoelectric layers. Inducing the displacement may include inducing a displacement at a frequency of between about 70 MHz and 80 MHz, wherein a penetration depth of an ultrasound signal emitted by the displacement is at least 0.6 cm. Inducing the displacement may include inducing a displacement at a frequency of between about 35 MHz and 45 MHz, wherein a penetration depth of an ultrasound signal emitted by the displacement is at least 1 cm. Inducing the displacement may include inducing a displacement at a frequency of between about 10 MHz and 20 MHz, wherein a penetration depth of an ultrasound signal emitted by the displacement is at least 4 cm.

For example, a method of sizing a stent for a blood vessel or lumen may include: positioning a combined imaging balloon catheter within the blood vessel or lumen, the combined imaging balloon catheter including: a catheter having a distal end with a flexible tip having an outer diameter of 3 French or less; an inflatable balloon assembly on the catheter proximal to the flexible tip; and an imaging assembly in the flexible tip distal to the inflatable balloon assembly, the imaging assembly including an array of piezoelectric micromachined ultrasound transducer (PMUT) sensors; imaging a portion of the blood vessel or lumen to be stented using the imaging assembly of the combined imaging balloon catheter without the inflatable balloon assembly being passed wholly through the portion of the blood vessel or wholly pulled back through the portion of the blood vessel or lumen, wherein imaging comprises applying a voltage between a plurality of electrode layers in each PMUT sensor of the array of PMUT sensors, wherein each PMUT sensor comprises a plurality of concentric multilayered stacks, each multilayered stack extending proud of a base layer over a cavity, further wherein each of the concentric multilayered stacks includes a plurality of piezoelectric layers, and wherein each piezoelectric layer is arranged between two electrode layers of the plurality of electrode layers; and inducing, from the applied voltage, a displacement that is a proportional to the applied voltage, a piezoelectric coefficient of a material forming the piezoelectric layers, and the number of piezoelectric layers; and determining a stent size based at least in part on the imaging of the portion of the blood vessel or lumen.

These and other examples, features and advantages are described herein.

### BRIEF DESCRIPTION OF THE DRAWINGS

Examples of the invention will now be described by way of example only with reference to the accompanying drawings, in which:

FIG. 1A shows an example of the imaging balloon catheter device;

FIG. 1B shows a detail of the imaging sensor assembly of the catheter of FIG. 1A;

FIG. 1C shows a cross section of the imaging sensor assembly of FIG. 1B;

FIG. 2 is an illustration of an example of a cross section of a catheter mounted transducer array;

FIG. 3 is an illustration of an example of a cross section through the catheter below the transducer array;

FIG. 4 illustrates a typical stent insertion procedure;

FIGS. 5A and 5B illustrate an example of the imaging balloon catheter, with detail of the transducer array area shown in FIG. 5B;

FIGS. 6A-G illustrate stages of a stenosis measuring procedure using an example of an imaging balloon catheter;

FIG. 7 shows an example of an individual cross-sectional image as displayed, imaging a stenosis within an artery;

FIG. 8 shows an example of a display output showing lateral and longitudinal cross sectional images of an artery with stenosis, and measurements output via the image processing system; and

FIGS. 9 and 10 illustrate an example of a combined imaging balloon catheter according to another example of the invention.

FIG. 11A illustrates a section view of an example piezoelectric stack showing a single piezoelectric layer.

FIG. 11B illustrates a perspective view and close-up view (inset) of a PMUT cell having concentrically arranged ring-shaped piezoelectric stacks similar to those shown in FIG. 1A.

FIG. 12A illustrates a section view of an example piezoelectric stack having two piezoelectric layers.

FIG. 12B illustrates a perspective view and close-up view (inset) of a PMUT having concentrically arranged ring-shaped piezoelectric stack of FIG. 2A.

FIG. 13 illustrates a stack of piezoelectric elements arranged in alternating polarity.

FIG. 14A is a graph showing calculated total displacement of a PMUT membrane achieved with different piezoelectric layer thicknesses.

FIG. 14B is a graph showing calculated total displacement of a PMUT membrane achieved with different bases layer thicknesses.

FIG. 15A is a graph showing calculated variation of the principal mode frequency of a single PMUT cell with respect to the radius of the piezoelectric layer.

FIG. 15B is a graph showing calculated variation of total displacement of the principal mode frequency of a single PMUT cell with respect to the radius of the piezoelectric layer.

FIG. 15C is graph showing calculated variation of the total displacement of the eigenmodes of a single PMUT cell have a particular cavity size.

FIG. 16A illustrates a simulation model based on calculated resonant modes and displacement fields for a ring PMUT with a single zinc oxide piezoelectric layer.

FIG. 16B illustrates a simulation model based on calculated resonant modes and displacement fields for a ring PMUT with a single aluminum nitride piezoelectric layer.

FIG. 17A is a simulation model showing total displacement of a ring array PMUT with a working resonant frequency of 13.54 MHz.

FIG. 17B is a simulation model showing total displacement of a ring array PMUT with a working resonant frequency of 42.92 MHz.

FIG. 17C is a simulation model showing total displacement of a ring array PMUT with a working resonant frequency of 78.98 MHz.

FIG. 17D is a simulation model similar to that shown in FIGS. 7A-7C, showing a cutaway region illustrating the displacement in the z-axis.

FIG. 18 is a graph comparing simulation results for calculated total displacement for resonant modes of a ring array PMUT having two piezoelectric layers and a ring array PMUT having a single piezoelectric layer of the same thickness.

FIG. 19 illustrates an acoustic field generated by an ultrasound transducer.

FIG. 20 shows a flowchart indicating a method for forming a PMUT device according to some examples.

FIG. 21A shows another example of a PMUT apparatus having concentrically arranged multilayered stacks configured as a continuous spiral.

FIGS. 21B-21F, showing examples of PMUT apparatuses having concentrically arranged multilayered stacks as described herein; FIG. 21B shows an example in which the concentrically arranged multilayered stacks are formed from a continuous rectangular (e.g., square) spiral. FIG. 21C shows an example in which the concentrically arranged multilayered stacks are formed from a continuous pentagonal spiral. FIG. 21D shows an example in which the concentrically arranged multilayered stacks are formed from a continuous hexagonal spiral. FIG. 21E shows an example in which the concentrically arranged multilayered stacks are formed from a continuous octagonal spiral. FIG. 21F shows an example in which the concentrically arranged multilayered stacks are formed from a continuous polygonal spiral.

FIG. 22A shows a linear array of PMUTs as described herein.

FIG. 22B shows a ring array of PMUTS as described herein, configured as a side-viewing ring array.

FIG. 22C shows a helical array of PMUTS as described herein, configured as a side-viewing ring array.

### DETAILED DESCRIPTION

Examples provide an imaging balloon catheter, control system and method of imaging during a catheterization procedure. Examples provide an imaging balloon catheter device having an imaging sensor assembly integrated into a balloon catheter. The balloon catheter can be based on known balloon catheter technology proven and currently used, for example balloon catheters used during percutaneous coronary intervention procedures (PCI).

An example of the imaging balloon catheter device comprising an elongate tubular body, an inflatable balloon element, and an imaging sensor assembly mounted proximate the inflatable balloon element. The elongate tubular body has a distal end for insertion into a vessel and a proximal end configured to remain external to the vessel accessible to a clinician and connectable to external components. The distal end is configured to support the inflatable balloon element and enable inflation and deflation of the balloon element, and the imaging sensor assembly mounted proximate the inflatable balloon element. The imaging sensor assembly is in data communication with an image processing system configured to process data output from the imaging sensor assembly and render for display.

Examples can be based on existing balloon catheter technology. Typical features of balloon catheters include an elongate tubular body having at least one inner lumen with an inflatable balloon element at or near the distal tip of the catheter, with a guide wire within an inner lumen of the catheter body for manipulation by a clinician or surgeon to control the catheter movement through the patient's vessels (i.e., arteries and/or veins) to position the distal end of the catheter proximate the site for treatment. The balloon is inflated, for example to dilate the artery, using a liquid delivered (and subsequently removed to allow deflation) via lumen in the tubular body. An example of a balloon dilation catheter is shown in U.S. Pat. No. 5,279,562. However, it should be appreciated that this is only one type of catheter that may be utilized.

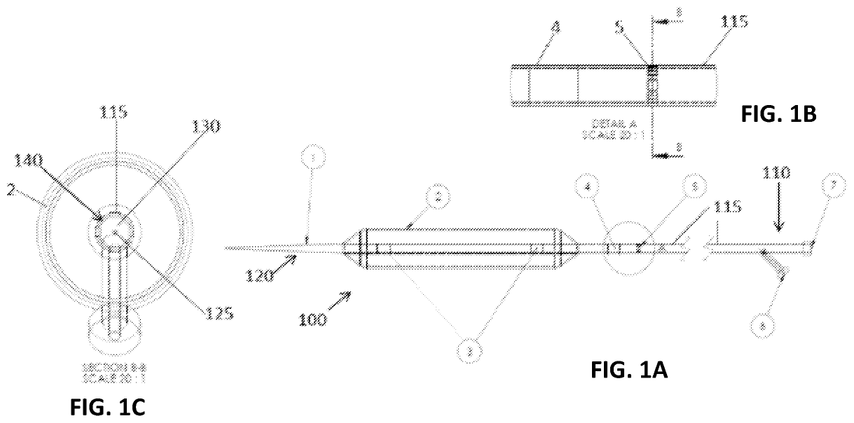

In example of the imaging balloon catheter as herein described an imaging sensor assembly is also carried proximate the balloon member. The imaging sensor assembly is mounted proximate the inflatable balloon element at the distal end of the tubular body. An example of an example of a balloon catheter suitable for a PCI procedure is shown in FIG. 1A. The imaging balloon catheter 100 has a narrowed tip 1, which tapers around the guidewire to be airtight. This helps maintain the pressure that is used to inflate the balloon 2. (The balloon is inflated with liquid). The balloon 2 is inflated to push out, for example to push out a stenosis. The balloon 2 is illustrated in an expanded position in the drawing of FIG. 1A, however it should be appreciated that the balloon hugs the catheter body when in the deflated state. The catheter body carries two radiopaque markers 3 to identify the start and end location of the balloon on a CT angiogram. In this example a further radiopaque marker 4 is proximate a sensor assembly, in this instance a transducer ring 5 used to image the artery. The transducer ring radiopaque marker 4 enabling the location of the transducer ring 5 to be identified on a CT angiogram. At the proximal end 110 of the catheter (accessible by the clinician in use) is an inflation port 6 used to inject liquid into the balloon catheter. This liquid is used to inflate and deflate the balloon. The catheter may also include a motor interface 7 for connection to a motor for performing the pullback of the device. This may also provide the connection point of the wires to the ultrasound transducer to connect to a processor for controlling the sensor assembly, and analysis and display of the imaging data.

The imaging sensor assembly is in data communication with an image processing system configured to process data output from the imaging sensor assembly and render for display. For example, to enable real time display of vessel lumen images to clinicians during the procedure. The processor may also be configured to output measurements such as minimum and maximum vessel diameter for an area of interest, length of an area of interest etc. The imaging sensor array can be configured to enable 360° imaging of the vessel lumen. Based on a combination of sensor assembly configuration and movement of the catheter 360° images of any desired second of the vessel lumen may be obtained and output to the clinical or surgical team. In some examples the imaging array may be configured to only image the interior of the vessel, i.e., the lumen and the surface of the lumen walls (the blockage). In alternative examples the imaging array may be configured to also capture data enabling imaging of the vessel walls and surrounding tissue, for example enabling indicating of tissue density, distinguishing diseased or injured tissue or blockage material from healthy tissue etc. Furthermore, it may be able to determine the composition of materials, to aid in the patients' treatment. The detail able to be provided by the imaging may be dependent on the sensor technology utilized in the sensor assembly. Additionally, processing techniques applied to the data captured using the imaging array can influence the amount of detail output via the system to the clinical team.

In an example the imaging sensor assembly is connected to an external control system via one or more leads through the catheter, the leads providing power, signaling control and data connectivity between the controller and the imaging array. The one or more leads may be carried within catheter lumen. A challenge for construction of the catheter to enable carrying the imaging sensor assembly, and also providing power supply and communication interface is maintaining small catheter size. Another challenge is ensuring sufficient lumen size for carrying the balloon inflation fluid. It should also be appreciated that balloon catheters are typically a consumable device, used once and discarded rather than sterilized and reused, and as such manufacturing cost minimization is also a consideration. In some of the exemplary examples discussed below simplicity in configuration of the sensor assembly and connecting leads is a consideration for manufacturing cost minimization, and other examples are envisaged.

In an example the imaging sensor assembly is configured to enable 360 degree imaging of the vessel interior for each imaging sample. For example, the imaging sensor assembly may be configured as a ring about the catheter body. This sensor may take multiple samples along the length of the vessel as the catheter is moved forward or backward along the vessel, with the data from multiple samples mosaicked together to provide a complete image along the vessel lumen.

In an example the imaging sensor assembly is a sensor array comprising one or more sensors. In the example shown in FIGS. 1A-C the sensor assembly is an array of ultrasound transducers 140 arranged concentrically around the catheter body 115 so as to not obstruct the central catheter lumen 130 and flow of inflation liquid.

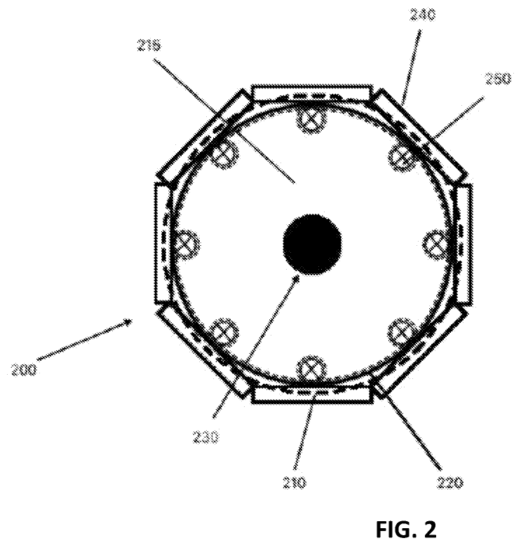

In an example the imaging sensor assembly uses ultrasound technology. The imaging sensor assembly can be an array of ultrasound transducers arranged in a ring around the body 115 of the catheter below the balloon element 2. An example of the sensor assembly is illustrated in FIG. 2, which is a diagram illustrating an example of a catheter mounted transducer array. FIG. 2 illustrates a cross section of the catheter through the transducer ring 200, showing the outer catheter wall 210, inner catheter wall 220, guidewire 230 within the inner lumen 215 which in this example also carries the fluid for inflation of the balloon. The region between the guidewire 230 and the inner catheter wall 220 is a hollow cavity where liquid is entered. The region between the inner and outer catheter wall can be solid.

In this example the transducers 240 are ultrasound transducers, arranged in a ring around the catheter body. In this example the transducers are arranged around the outer catheter wall, embedded in the catheter wall and exposed on the outer surface. In the example shown the outer surface of each transducer 240 is flush with the outer catheter wall 220. However, the transducers 240 may also protrude outside the catheter wall 220.

The exposed outer surface of the transducers is to allow direct contact with blood in the vessel which can maximize imaging sensitivity. It should be appreciated that ultrasound is a pressure wave sensing technology, so having direct contact between the sensors and blood (which is the pressure wave transmission medium) signal attenuation is minimized, which may improve detection sensitivity. This may also improve signal to noise ratio. However, there may also be examples where the transducers are not directly exposed to the blood. For example, an example may include sensors located within the balloon to allow imaging during stenting. Signal processing may be utilized to distinguish data for the vessel from the balloon. In another example a thin coating may be applied over the transducer array. This may reduce the transducer sensitivity but be required due to other considerations. One example of a reason for coating the transducer array may be to minimize risk of contamination and to ensure sterility. A coating may also be used to minimize risk of abrasion or other damage to vessel walls during catheter insertion and extraction, for example for examples design for use in the brain, or for pediatrics or neonates, where there may be an increased risk of damage to vessel walls.

From this cross section one can see that in this example eight sensors have been used, however other examples may use, 4, 7, 10, 16, 32, 50, 64 etc. sensors, any number of sensors may be used. More sensors may improve imaging resolution and/or reduce imaging processing requirements. However, additional sensors may increase device cost. Examples may use any number of sensors, limitations to number of sensors being based on multiple factors including: size, cabling, processing requirements, cost etc. with the example designer balancing these as a matter of design choice. Any sensor technology capable of imaging a vessel wall without requiring physical contact with the wall may be suitable.

The transducers 240 can be any ultrasonic component. Two examples of suitable transducers are CMUT's (capacitive micromachined ultrasound transducers) or piezoelectric. However other transducer technologies may be used. The key considerations for choice of transducer technology being any one or more of: size, sensitivity, suitability for use within the human body, and cost.

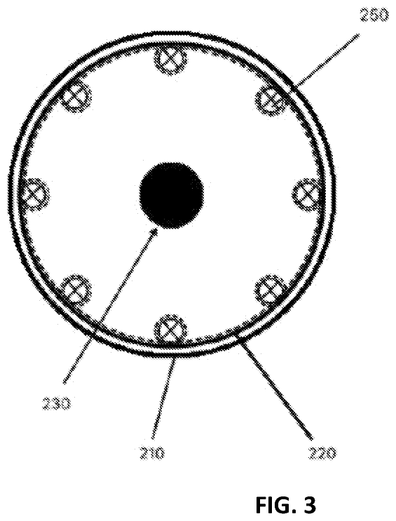

The example shown in FIG. 2 also illustrates the connectors 250 (in this example wires) connecting each transducer, providing power, signaling and data connectivity to the external controller and image processor. FIG. 3 shows a cross section of the catheter taken between the transducer array at the distal end and the proximal end of the catheter body where connection can be made to the external system components controlling the image sensing and image processing. In an example the wires are coaxial with an innermost signal wire and an outermost ground wire. A ground wire is optional. Although the ground wire is not strictly necessary removing the ground wire can negatively impact signal integrity. Other electric conductor types are possible, for example biocompatible strip conductors, cables, thin film conductors, etc. Some examples may utilize technology other than electrically conductive connectors, for example optical fibers, for imaging signals.

In an example the connectors/wires may not be fully embedded in the catheter wall and so protrude into the inner hollow section. Regulatory requirements may stipulate that any exposed wires should be insulated using a biocompatible insulating material. If the conductors are of biocompatible material, then such insulation may not be required. If the wires are fully embedded within the catheter wall may not need bio compatible insulation. The connector type and configuration may vary between examples based on regulatory requirements and/or design choice.

In the example shown each transducer of the array is connected separately, with one cable for each transducer and each transducer driven directly by the external controller. This simplifies the wiring required to be carried within the catheter body and avoids any requirement for additional control electronics to be incorporated into the distal end assembly of the catheter. It should be appreciated that this connection regime, by minimizing the complexity, may enable cost efficient production of the disposable catheter components. Further, a simplistic catheter operation may also reduce quality assurance testing requirements for the catheter mounted electronics.

In the example illustrated in FIG. 3 each wire 250 extends individually through an outer lumen formed by the inner 220 and outer 210 catheter walls. However, in an alternative example the wires may be grouped to form a bus extending through the catheter. Alternative examples may include a microelectronic switching circuit at the distal end of the catheter to enable serial connection and sampling of the image signals via a serial bus. Design for such an example may take into consideration balancing material costs with production complexity. This example use of serial sampling may limit the maximum imaging speed. However, as the catheter pull back rate is limited based on physical patient safety considerations (for example to around 2 mm/s), limiting imaging speed may not result in any detriment to device performance or image quality.

The imaging balloon catheter system includes a processor configured to analyze and display image data. The processor can be configured to monitor movement of the catheter as the catheter is extracted. The movement data is input to analysis of sampled image data. For example, the length of a stenosis can be determined based on the movement and sampling rate of the transducer array and detection of the start and end of the abnormality (stenosis).

The system may optionally include a mechanism for controlling extraction of the catheter, whereby the catheter can be extracted at a pre-set pull back rate. Such devices are known and often used by clinicians to ensure controlled pull back and also free the clinician to focus on other aspects of the procedure and/or patient monitoring during the catheter pull back. In an example the imaging controller may control sampling based on pull back rate. In some examples the sampling controller may be integrated with the pull back motor controller and synchronize image sampling with the pull back.

In alternative examples the imaging processor may be configured to monitor movement of the catheter is based on marker detection, for example, through integration with a CT angiogram system. For example, the image cross sections can be pieced together based on detecting the location of the transducer array at the time the image was sampled. The system may be configured to compare a transducer marker position on a CT scan with a scale to determine the position. In another example the distal end of the catheter may carry a plurality of evenly spaced markers (i.e., optically or electromagnetically detectable markers) and the processor configured to count the number of markers to determine distance between samples, or count how many markers go past a detector in a second to indicates effective pullback speed. In another example the imaging balloon catheter may include motion sensors such as an accelerometer mounted on the catheter body to enable monitoring of movement of the catheter. Other types of motion sensors that may be used include velocity meters, gyroscopes, optical sensors etc. Motion monitoring may utilize a combination of different sensors and technologies. The Imaging processor may be configured to monitor the motion sensors to determine catheter movement for input to sampling control and or image processing for display.

It will be appreciated that detection motion of the catheter may be advantageous for non pullback methods of imaging. This is advantageous as 3D artery reconstruction may be performed passively, i.e., as a clinician inserts catheter, rather than on retrieval. This may also eliminate the need for a motor, lowering the complexity of using the device and lessening the setup time.

It will also be appreciated that motion detection is not limited to a single method—i.e., accelerometers or marker detection. For example, sensor fusion algorithms which use data from multiple sources to reduce the measurement error may be used in the application to know precisely where a cross sectional image was taken.

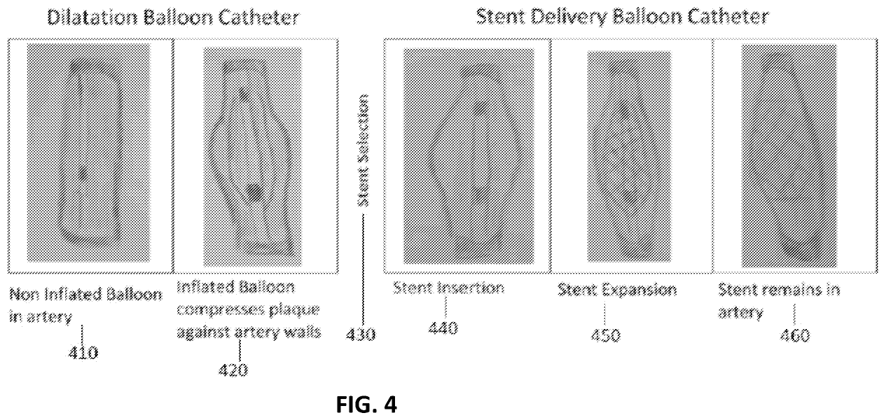

FIG. 4 illustrates a typical stent insertion procedure, part of a Percutaneous Coronary Intervention (PCI). An Angiogram procedure often takes place prior to a PCI. This procedure identifies that a blockage is present and requires a stent. In the initial steps of the PCI procedure a sheath catheter can be inserted into the body through either the radial or femoral artery and travels into the coronary artery. All other catheters and guidewires can then travel through the sheath catheter. Radio Opaque dye is released, an external x-ray is taken to map the arteries with and to locate the blockage. A guidewire is passed through the blockage to guide positioning of catheters for the stenting procedure.

For a typical stent insertion procedure in step 410 a balloon catheter (deflated) is inserted into the artery, after the guidewire is positioned, to travel along the guidewire to a position at the site of stenosis (plaque build-up). In step 420 the balloon is inflated to compress the plaque against the artery walls to widen the passage for blood flow. The balloon catheter is then removed and a stent chosen 430 for the next part of the procedure. Often the stent choice is made based on a best guess and limited information available from a CT scan. The stent is then inserted 440, carried on a balloon catheter, and the balloon inflated to expand the stent 450. The balloon is then deflated 460 and the catheter removed, leaving the stent in place holding the artery open. The surgeon/clinician then checks the angiogram to confirm placement of the sent and determine whether the stent secured the whole blockage. As discussed in the background section the stent choice step is often performed with limited understanding of the geometry of the artery blockage, leading to errors. It is not uncommon for a further stents to be required. Examples of the present imaging balloon catheter are designed to address this problem.

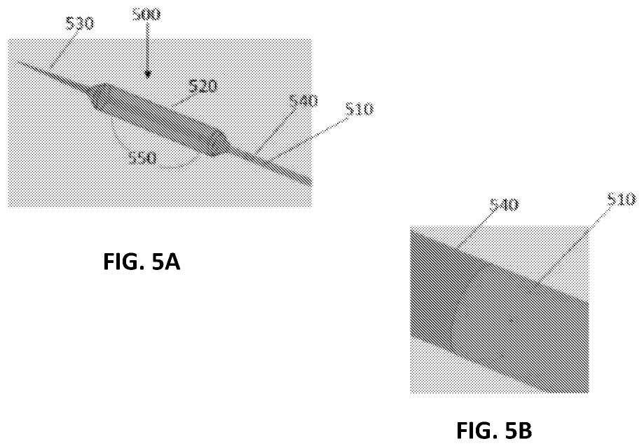

In this example the imaging balloon catheter has an imaging sensor assembly comprising a ring of transducers around the catheter near the balloon. An example of the imaging balloon catheter 500 is illustrated in FIG. 5A with FIG. 5B showing detail of the transducer region. In the example shown the transducer ring 510 is on the opposite end of the balloon 520 to the catheter tip 530—this configuration removes the requirement to have signaling wires extend through the balloon section 520 of the catheter. An additional radiopaque marker 540 is provided proximate the transducer ring 510 to enable the transducer ring 510 position to be distinguished on a Computed Tomography (CT) angiogram image.

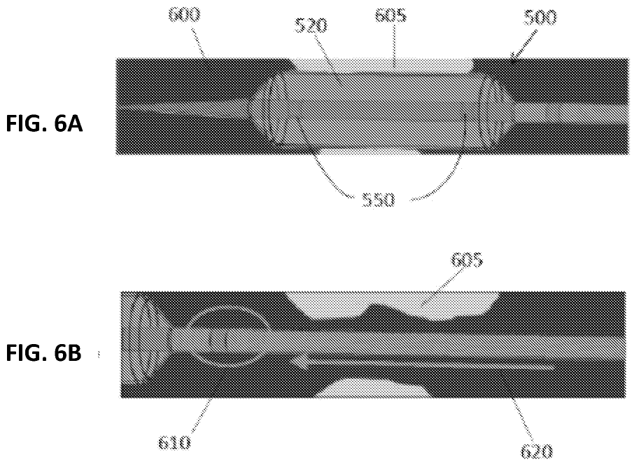

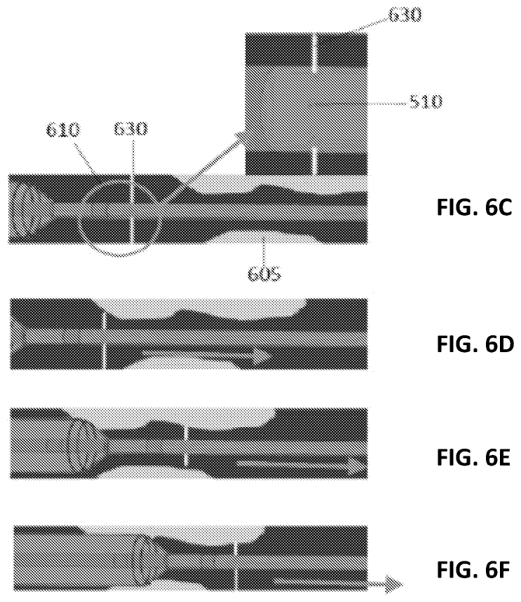

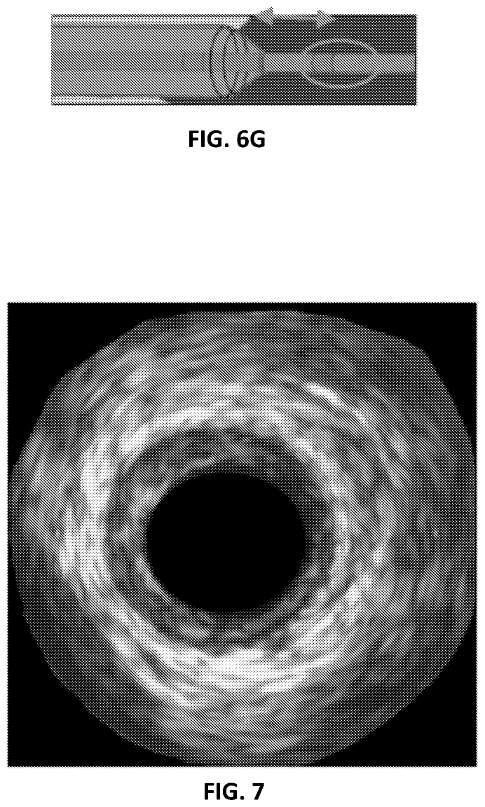

An example of a vessel measuring procedure, which can be performed as part of a PCI stenting procedure, is illustrated in FIGS. 6a-g. Similar to as shown above in step 410 the imaging balloon catheter 500 is inserted into the artery 600. Positioning of the imaging balloon catheter 500 is guided using the radiopaque markers 550 on a CT scan, to position the balloon section of the catheter 500 proximate the blockage 605 in the artery 600. The balloon 520 is then inflated at the site of the blockage 605 by pumping saline down the catheter to expand the balloon 520, as shown in FIG. 6A. The balloon will be inflated at the point of the stenosis, to push the plaque up against the artery walls to restore blood flow. The balloon is then deflated.

The imaging balloon catheter is then moved forward past the blockage 605 so that the ring of ultrasonic transducers 510 is moved to the most distal component of the blockage, as shown in FIG. 6B. The placement of the transducer array 510 proximate the blockage 605 can be visualized on the CT-scan by the radiopaque marker 610 placed near the location of the sensors. In this example the radiopaque marker is a gold marker, however other marker types may be used, for example a platinum marker or platinum coil. The catheter can be manually moved by the clinician while watching the position of the transducer marker on a CT scan so that the transducers are positioned just past the proximal end of the stenosis. It should be noted that in the drawings the balloon is not shown deflated to enable the relative position of the balloon and sensor array to be more apparent.

Once the sensor array 610 is positioned ahead of the far end of the blockage 605, then sensing performed as the catheter is pulled back. As show in in FIGS. 6C-F the sensors sample image data radially (the yellow line) 630 as the catheter is pulled back. The clinician may input a trigger to start imaging, or the system may monitor the position of the transducer array and automatically trigger imaging.

In an alternative example the imaging processor may be configured to transmit periodic ultrasound pulses as the catheter is moved forward and detect the far end of the blockage. This may be used as an alternative or in addition to CT scan based placement in some examples. For example, the imaging processor may output vessel diameter data to allow the clinician/surgeon to verify that the transducer array has moved past the blockage. Alternatively, the imaging processor may be configured to assess whether the blockage has passed based on the signals and output an indicator—for example a visual or auditory indicator—once the transducer has moved past the blockage. For example, this may be based on measurement of the diameter of vessel. Alternatively, the data received via the sensor transducer may enable the processor to distinguish the stenosis from healthy vessel wall tissue, for example based on texture or density of the vessel lumen, to detect the end of the stenosis. It should be appreciated that this method may make the system more computationally complex but may also improve accuracy or allow measurement of stenosis that is not apparent on the CT scan. In an example the transducer may also be configured to image the stenosis as the catheter is pushed forward past the blockage.

FIG. 6C shows the transducer array positioned past the blockage and activated, to transmit ultrasound pulses and receive reflected signals, which are transmitted to the processor for rendering the image. In the example shown the transducers transmit ultrasound pulses radially 630, however, examples where the transducers are angled forward or backward are also envisaged. In the example shown in FIGS. 6D to 6F a pull back motor is used to control pull back of the catheter. The motor is turned on to pull back the catheter distally along the stenosis at a known speed. The ultrasound data is sampled as the catheter is pulled back. The signals generated are then electrically sent to the software component of the device which will output both stent length and stent diameter that the patient requires.

In an example as the transducer moves along the stenosis, individual cross-sectional images every 0.25 mm are displayed on external user interface, and example of a cross section is shown in FIG. 7. The motor is shut off once the yellow transducer marker passes the distal end-point of the stenosis, as illustrated in FIG. 6G. In this example the samples are taken at 0.25 mm intervals as this is based on cardiac stent sizing. The sample rate may vary between examples (for example 0.05 mm to 10 mm) depending on the purpose of the measurement. In an example for neural vessel imaging and stenting a sample rate of 0.05 mm to 0.2 mm may be appropriate, for vascular stenting a sample rate of 0.2 mm to 10 mm may be used depending on the vessel and stent size options for the vessel in question. In some examples the imaging processor may be configured to operate with different types of imaging balloon catheter devices for different applications, and vary sampling rate based on the application and device used.

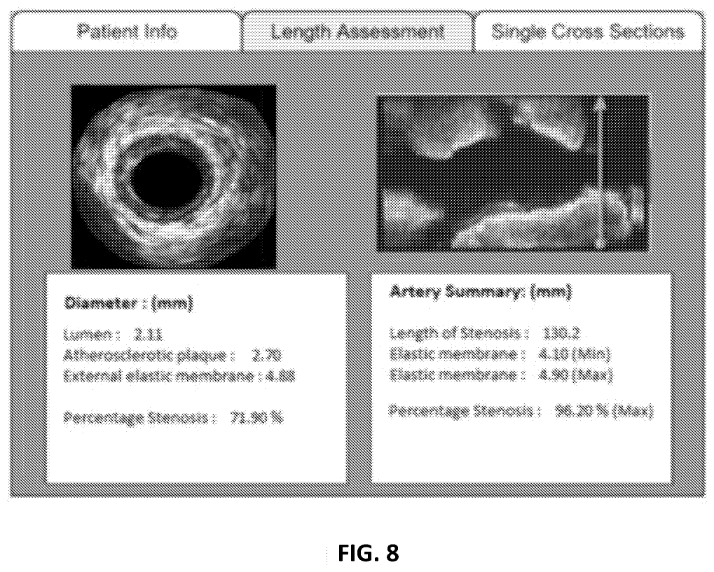

Full 2D cross-sectional image of the stenosis as well as the associated measurements required for accurate stent selection are displayed on external user interface. FIG. 8 shows an example of a display output showing lateral and longitudinal cross sectional images of an artery with stenosis, and measurements output via the image processing system. In particular the system outputs a measurement of the length of the stenosis and lumen diameter. The system can also output elastic membrane parameters. For example, these parameters can provide information regarding innermost diameter and outermost diameter of the vessel in the obstruction/treatment region to enable the clinician to determine diameter and length required to be supported by an implanted stent. This data can be used by the surgeon/clinician for stent selection. Having accurate measurement of the length of the vessel requiring stenting support and also the lumen diameter can enable the surgeon/clinician to choose a suitable stent more reliably.

In some examples the processing system determines and outputs a treatment area length based on the sampled image data. The treatment area length is determined based on identification of a start and end of a vessel wall abnormality based on the image data, and calculation of a distance between the start and end. This may be calculated automatically by the system, or the system may output sufficient data and scaling information to allow a clinician and/or surgeon to make a choice, for example displaying a scale in conjunction with imaged data.

After imaging to enable stent selection the imaging balloon catheter device may be removed and a stent delivery catheter, comprising a balloon catheter loaded with the chosen stent used to insert the chosen stent a shown in FIG. 4. Examples may include an imaging balloon catheter also configured to enable correct stent placement over a lesion/stenosis. Current stenting procedures typically use two balloon catheters, one for initially clearing the vessel obstruction and a second for delivering the stent to the treatment area and expanding the stent into place over a legion/stenosis to support the vessel against occlusion. It is envisaged at both such balloon catheters may include an imaging sensor assembly as described. For example, an advantage of a stent delivery catheter including the transducer array as described can include the ability to utilize the imaging for positioning verification before stent expansion, or enabling post insertion imaging of the stent for example to confirm placement and expansion of the stent. For example, a stent is loaded over the balloon of a stent delivery catheter. With a transducer array located behind the balloon as shown in FIG. 5A the transducer array may be used to ensure correct positioning of the balloon relative to the end of the stenosis before expansion. For example, similar to a shown in FIG. 6G the distal catheter tip and balloon portion can be inserted through the stenosis and pushed forward to a point where the end of the stenosis is detected by the transducer array. Based on the distance between the transducer array and end of the stent, the catheter can be drawn back to position the end of the stent aligned with the end of the stenosis. The balloon can then be inflated to expand the stent. The transducer array may then be used to image the stented artery using the procedure as described above.

Some examples may be configured to for manual movement of the catheter. During imaging the movement of the catheter is monitored to provide movement data characterizing movement of the catheter. The image data acquired form the sensor array, for example by sampling image data periodically during pull back, can be processed taking the moment data into consideration. For example, using a pull back motor the catheter may be pulled back at a fixed rate and the sampling rate synchronized to this fixed rate, say pulling back at approximately 0.5 mm/s. However, during manual pull back the movement rate will typically be variable, but the sampling rate may be fixed. The movement data can be utilized to determine the linear distance moved between each sample and utilized when rendering images. The movement data may also enable angular distance between each sample to be determined imaged rendered for display with orientation adjusted accordingly.

It should be appreciated that for a transducer ring example the angular distance between each sample may not be of significant interest. However, in an alternative example where the imaging sensor is a linear array or helical array extending along the catheter body and rotated to ensure 360° imaging then relative angular/rotational distance between each sample can be critical for accurate image rendering. Methods for monitoring manual movement of the catheter may include using one or more accelerometers to detect longitudinal and rotational movement of the catheter. As the catheter body, although laterally flexible for movement through the vessel lumen, will typically have a fixed length and be resistant to twisting along the catheter length, so that accelerometers mounted at the proximal end of the catheter body may provide an accurate indication of longitudinal and rotational movement of the distal tip relative to the vessel walls.

The sampled image data is processed using movement data to render and display images of the vessel lumen, for example as shown in FIG. 7. This image data can be used to determine appropriate stent length and diameter as the rendered image data can accurately show the length of the blockage.

In an alternative example the imaging sensor assembly may comprise a plurality of sensors arranged in a linear array. The linear array may extend longitudinally along one side of the catheter and the catheter rotated to enable 360° imaging. Alternatively, the longitudinal array may be arranged in a spiral around the catheter body. One disadvantage of longitudinally extending arrays may be that insertion the catheter significantly past an obstruction or treatment area is required to enable imaging. In some examples the imaging assembly may be integrated with the balloon element, for example one or more transducer strips along the outside of the balloon, or an array carried on the catheter body internal to the balloon. Such examples may use sensor technologies other than ultrasound. For example, alternative imaging technologies may utilize electrical capacitance or resistance tomography, or electromagnetic induction tomography (eddy current) techniques. Ultrasound transducers within the balloon element may suffer from too much signal attenuation or interference form the balloon itself for effective imaging, with the signal to noise ration of the ultrasound transducers maximized with good blood contact. However, an alternative sensing technique such as electromagnetic induction tomography, or alternatively new sensor technologies, may not suffer from these drawbacks, thus enabling a broader range of device examples.

Optical imaging techniques may also be used. Examples of optical sensor methods that may be used include OCT (optical coherence tomography), Near IR spectroscopy (NIRS), Video/photography. These examples use imaging sensor assemblies located near the balloon at the distal tip of the catheter as described above. These methods may however use fiber optic connection to the imaging processor rather than electrical connection. The sensor type and image processing are dependent on the imaging technique. For example, such examples may utilize sensing arrays comprising light transmitters (i.e., LEDs or optical fibers connected to an external light source) and receivers (CCDs or receiving optical fibers). For optical fiber-based examples it should be appreciated that the distal end of the may fibers effectively form the imaging assembly and the length of the fibers form the communication path to the processor. Optical imaging techniques may use visible light or specific wavelengths/frequencies or spectrum ranges, for example near infrared.

Optical coherence tomography (OCT) utilizes coherent light to capture high resolution images from within optical scattering media, in this application the vessel wall acts as the scattering media. Similar to intravenous ultrasound (IVUS) examples, OCT based imaging provides artery cross sections which can be used to identify a stenosis. Near infrared spectroscopy (NIRS) provides composition of measure material and this is used as the basis for detecting the presence of stenosis. The amount of material detected enables calculation and simulation of the artery cross section. Video and photography techniques utilize visible light to image the artery wall. Visible light can penetrate up to a few mm of tissue. Using image processing techniques, the composition of the vessel wall can be determined (i.e., presence of a stenosis and its thickness).

Examples of the imaging balloon catheter are configured for imaging of the vessel lumen. An objective is to image the entire cross section to look for the presence of a stenosis this is achieved either with a single transducer or array that rotates (either by rotation of the catheter or via a drive shaft) or via multiple transducers arranged for 360° imaging (ring structure). Thus, for examples using ultrasound technology, B-mode (brightness mode) imaging techniques are used. A-mode can provide a graph of ultrasound echo strength vs time and is only applicable to a single transducer in a fixed position. B-mode (brightness mode) uses the same data as A-mode (echo strength) but instead in an image where white pixels represent a strong echo and black pixels represent absence of an echo. The rendered image contains data from all transducers. B-mode is useful for displaying information to a clinician in a manner that is easily interpreted.

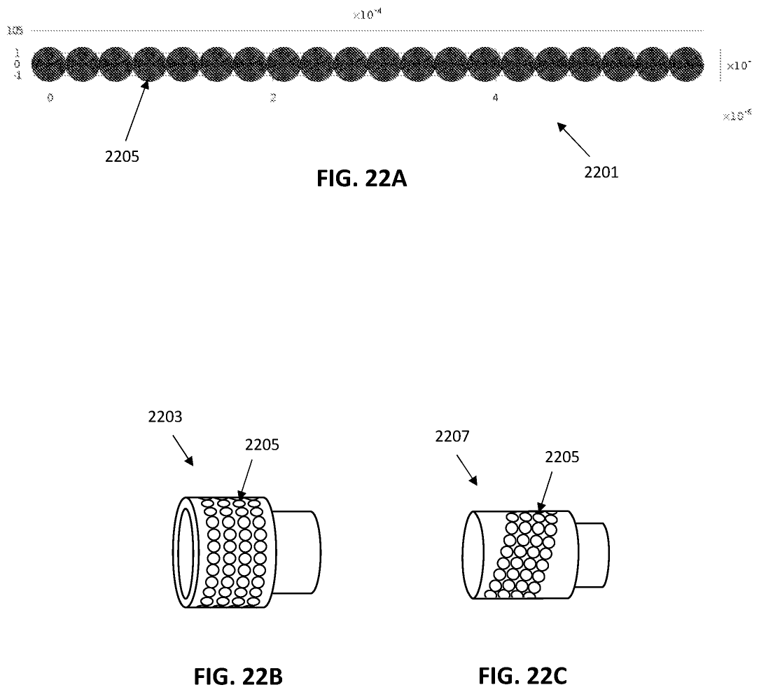

Examples utilize b-mode transducer array structure and processing techniques so that the entire artery can be checked for stenosis, for example, using multiple ultrasound transducers in a ring structure (e.g., FIG. 22B), helical array (e.g., FIG. 22C), or rotatable linear array (e.g., FIG. 22A) to enable capturing of multiple samples covering 360°.

In an example the sensor assembly comprises a ring of micro-sized ultrasonic transducers placed in a ring proximate the balloon. The sensor assembly may be located at the probe tip rather than below the balloon. Alternative placements of imaging sensor assemblies are envisaged enabled by reduced sensor size and alternative technology which may be available currently or become available in the future. All such alternatives are envisaged within the scope of the present imaging balloon catheter.

It envisaged that a remotely powered and wireless communication sensor assembly may be technically feasible. However, such an example may be impractical for a number of reasons, for example this may require increased size of the sensor assembly using current technology, or increase costs for the consumable catheter component, this may limit the operating time (due to short battery life). Further it is often desirable or even essential to minimize (or avoid altogether) radio frequency transmissions in operating environments.

It should be appreciated that the combination of vessel imaging and expansion being enabled in one device may significantly reduce the equipment and time required for catheterization procedures. Examples of the imaging balloon catheter have extensive commercial application in the medical industry. Primarily, examples would be used in Percutaneous Coronary Intervention procedures. Other applications may include neurological stenting procedures and vascular stenting procedures.

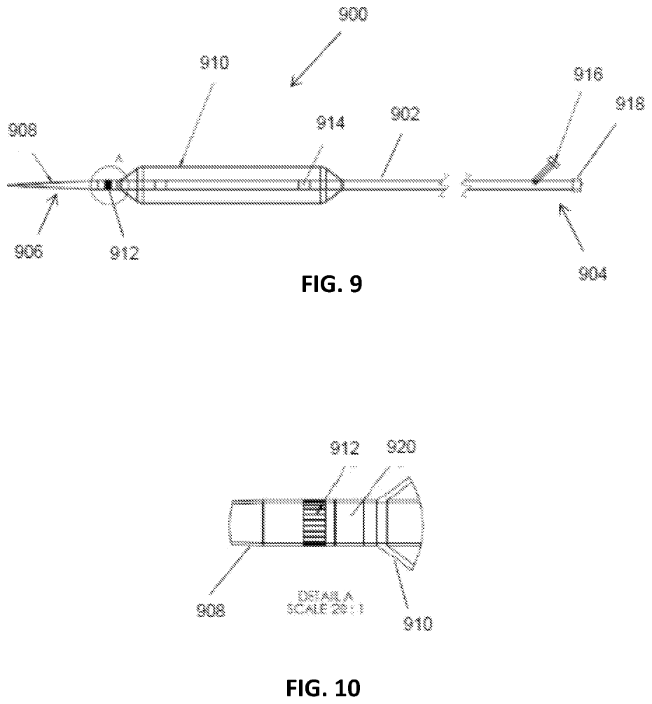

FIGS. 9 and 10 illustrate an example combined imaging balloon sensor 900 according to one example of the present invention. The combined imaging balloon catheter 900 may comprise a catheter 902 having a proximal end 904 and a distal end 906 with a flexible tip 908. An inflatable balloon assembly 910 may be provided on the catheter 902 proximal to the flexible tip 908. An imaging assembly 912 may be provided in the flexible tip 908 distal to the inflatable balloon assembly 910. The flexible tip 908 may narrow distally away from one or both of the inflatable balloon assembly 910 and the imaging assembly 912. The flexible tip 908 may be configured to have a length and flexibility sufficient to house the imaging assembly 912 for imaging a portion of a blood vessel or lumen to be dilated or stented, but insufficient to disturb or puncture a wall of the blood vessel or lumen.

The catheter 902 may have an outer diameter of 3 French or less. The catheter 902 may have the torquability and flexibility of conventional balloon catheters. In addition, the catheter 902 may have the typical functionality of conventional balloon catheters. For example, the catheter 902 may comprise a balloon radio-opaque marker 914 to aid the clinician in identifying where the balloon is located within the patient's arterial system with the aid of a coronary angiogram. A coronary angiogram is a clinician's primary method of guiding catheters within a patient. The catheter 902 may further comprise an inflation port 916 to enable the clinician to inject/remove saline solution to inflate/deflate the balloon, and a motor interface 918. The inflation port 916 may be positioned far away from motor interface 918 as to not interfere.

The motor interface 918 may be configured to interface with the electronics and processor of the system to construct a 3D model from cross sectional images at known locations where the cross sectional images were taken. A pullback motor may provide a known rate and direction of motion for the catheter 902 so that the cross sectional images have a known relative location from one another. An external pullback motor (not shown) may be physically connected to the catheter 902 to achieve this.

Similarly, the sensors may be connected to an external electronic unit to provide power, trigger imaging and receive the imaging data. The imaging data may, for example, comprise voltage signals that need to be sampled by the external electronics and converted into an image. The inflatable balloon assembly 910 may comprise a compliant or non-compliant balloon. Compliant balloons expand with increased pressure. This relationship can break down however when the blockage is resistant particularly when it is calcified. Non-compliant expand to a set size. The balloon may have one or more uses, including but not limited to, being used to push the blockage outward (i.e., dilatation balloon catheter) thus restoring blood flow, expand a stent (i.e., delivery balloon catheter), or deliver a drug (i.e., drug coated balloon catheter). Balloons may be provided in different sizes and different pressure ratings.

The imaging assembly 912 may, for example, comprise intravascular ultrasound (IVUS) sensors. The IVUS sensors may, for example, comprise an annular or non-annular array of piezoelectric micromachined ultrasound transducers (PMUTs). The IVUS sensors may be small enough to be incorporated into a standard balloon catheter 902 having an outer diameter of 3 French or less. The sensors may be arranged in an annular or non-annular array. The sensors may have unique material and acoustic properties compared to current ultrasound sensors used in the coronary space. This may provide flexibility to alter sensor properties to explore several avenues for improved imaging capabilities (including resolution and tissue penetration), as well as sensor manufacturability.

The IVUS sensors may be arranged in an annular arrangement with sensors displaced along the length of catheter (helical, 2 rings with sensors offset between rings etc.).

The IVUS sensor array of the imaging assembly 912 may advantageously be located on the tip side of the balloon (i.e., distal to the balloon). This allows the imaging component to pass into a blockage without the balloon being wholly passed through or wholly pulled back through the blockage, thereby potentially disturbing the blockage. This design feature may ensure the safety of the patients whilst imaging. There is naturally apprehension in passing a used balloon—particularly if it is incompletely wrapped—through a recently deployed stent for fear of strut disruption. In addition, there is also apprehension in pulling a used balloon wholly back through a blockage for fear of disrupting or dislodging the blockage.

A radio opaque sensor marker 920 may be provided proximal to the imaging assembly 912. Similar to the balloon catheter marker 914, the sensor marker 920 may show clinicians where the sensor array is located within a patient's arterial systems with aid from a coronary angiogram.

Examples of the combined imaging balloon catheter may be used in a method to size a stent for a blood vessel or lumen. The method may comprise imaging a portion of the blood vessel or lumen to be stented using the imaging assembly of the above combined imaging balloon catheter without the inflatable balloon assembly being passed wholly through the portion, and then being pulled wholly back through the portion of the blood vessel or lumen. The stent size may then be determined based at least in part on the imaging of the portion of the blood vessel or lumen.

Examples of the present invention provide a combined imaging balloon catheter that is both specifically and generally useful for PCI procedures and stent sizing.

Any of the catheter devices described herein can include PMUTs that are small enough to be integrated into the catheter device, yet can that operate at high frequencies and with high penetration depth for a given applied voltage to provide high resolution ultrasound images.

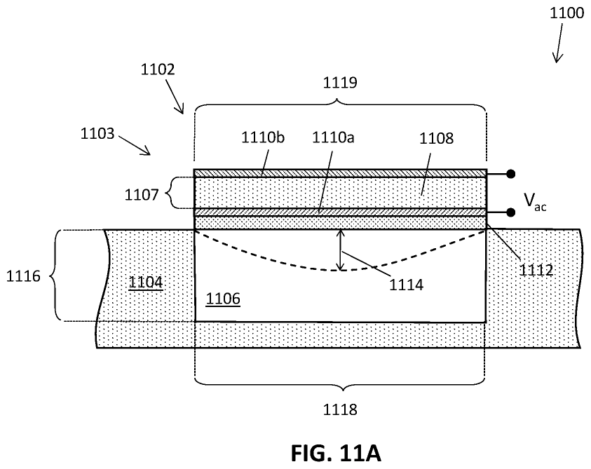

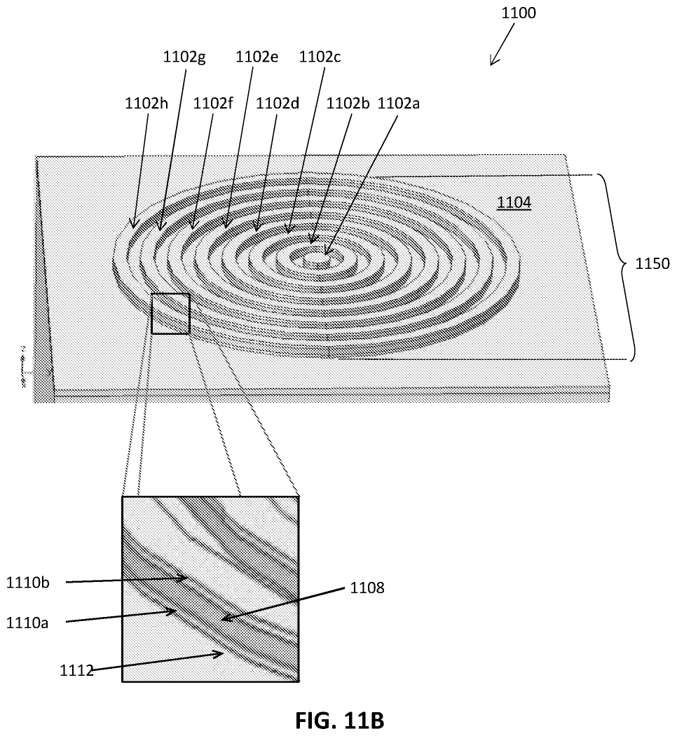

FIG. 11A shows a section view of a portion of an example piezoelectric stack 1102 as part of a PMUT device cell 1100. Note that a cell 1110 may be referred to herein as a PMUT or PMUT sensor. The piezoelectric stack 1102 includes a multilayer stack 1103, which acts as a vibrating membrane (also referred to as a diaphragm) formed over a corresponding cavity 1106 in a substrate 1104. The cavity within the substrate may be formed, e.g., using an etching process during the fabrication of the PMUT device. The multilayer stack membrane can include a first piezoelectric layer 1108 made of a piezoelectric material. The piezoelectric layer can be situated between a first electrode layer 1110a (e.g., bottom electrode layer) and a second electrode layer 1110b (e.g., top electrode layer) that are operationally coupled to a power source (e.g., AC current source). A direction of polarization of the piezoelectric layer can be arranged parallel to a height 1107 of the piezoelectric layer. The multilayer stack can also include a base layer 1112 between one of the electrode layers (e.g., bottom electrode layer) and the substrate. When voltage is applied on the electrode layers, the piezoelectric layer converts the electrical energy to mechanical energy by vibrating at an excitation frequency. The excitation frequency can depend, in part, on the geometry of the piezoelectric cell. This vibration causes the suspended multilayer stack membrane to deflect 1114 within the cavity of the substrate, thereby generating movement and force (e.g., movement in a direction that is perpendicular to the substrate/membrane of the device). In some examples the movement may be linear. In some examples the movement may not be linear (e.g., the base layer 1112 can add rigidity to the membrane during the vibration, thereby affecting the degree of deflection of the membrane. As will be described in greater detail below, in general, any of these apparatuses may include multiple piezoelectric layers, although only one is shown in FIGS. 11A-11B.

According to some example, the PMUT devices can include a number of ring-shaped piezoelectric stacks that are concentrically arranged (ring array), which can provide better focusing of the PMUT compared to simple round or rectangular PMUT structures. FIG. 11B shows an aerial view of the piezoelectric transducer 1100 showing a concentrically arrangement of piezoelectric stacks 1102a, 1102b, 1102c, 1102d, 1102e, 1102f, 1102g and 1102h on the substrate 1104. In this example, the piezoelectric stacks 1102b-1102h are ring-shaped and arranged concentrically about a center piezoelectric stack 1102a having a circular shape. Each of the piezoelectric stacks 1102a-1102h can include the features of the piezoelectric stack 1102 described above with respect to FIG. 11A. Each of the piezoelectric stacks 1102a-1102h includes a multilayer stack membrane, which includes the piezoelectric layer 1108, electrode layers 1110a and 1110b, and base layer 1112.

In general, these apparatuses may include one or more cavities. For example, in some examples, all of the multilayered stacks forming the cell of the apparatus may be arranged over a single cavity within the substrate. For example, the ring-shaped piezoelectric stacks 1102b-1102h can be arranged over a single cavity, and the circular-shaped piezoelectric stack 1102a is arranged over the same cavity. The outer edge of the outer ring of the piezo electric stacks may positioned at the perimeter of the cavity.

Simulations show that a PMUT device having a ring array arrangement may provide improved performance in terms of vibration frequency compared to a PMUT device having a simple circular or rectangular piezoelectric cell of the same size (e.g., diameter), which can allow for better imaging resolution with the same penetration depth. For example, simulation results indicate that a ring array PMUT can provide higher vibrational amplitudes for 1 V driving voltage compared to a PMUT having a single circular cell. Examples of such simulations are described further below.

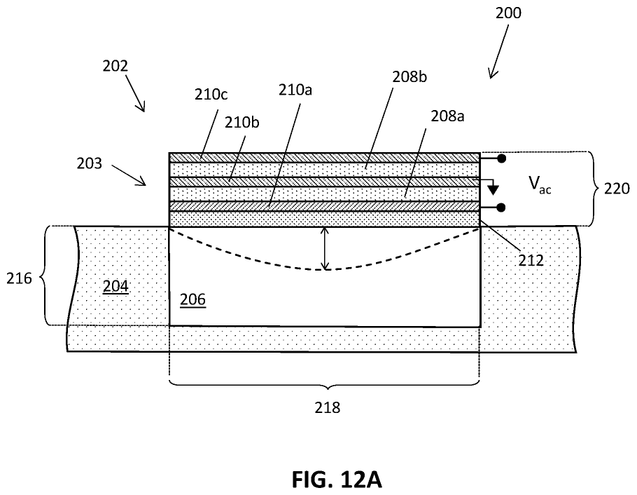

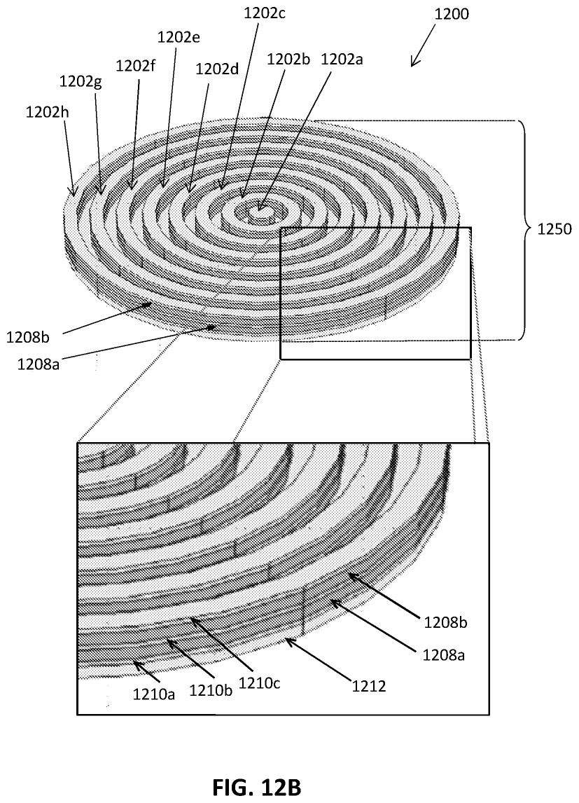

According to some examples, the PMUT devices include multiple piezoelectric layers. Multiple piezoelectric layers may be useful in ultrasound imaging applications since stacked piezoelectric layers can increase the vibrational amplitude and penetration depth of ultrasound in tissue compared to a PMUT having a single piezoelectric layer. For example, simulations have shown a PMUT having stacked piezoelectric layers in a ring array arrangement are shown to have a 150-times higher vibrational amplitude compared to a PMUT having a single piezoelectric layer in a ring array arrangement. FIG. 12A shows a section view of a portion of an example piezoelectric stack 1202 as part of a PMUT device 1200. The piezoelectric stack includes a multilayer stack membrane 1203 formed over a corresponding cavity 1206 in the substrate 1204. In this example, the multilayer stack membrane 1203 includes a first piezoelectric layer 1208a (e.g., bottom piezoelectric layer) and a second piezoelectric layer 1208b (e.g., top piezoelectric layer). The first piezoelectric layer 1208a can be situated between a first electrode layer 1210a (e.g., bottom electrode layer) and a second electrode layer 1210b (e.g., middle electrode layer), and the second piezoelectric layer 1208a can be situated between the second electrode layer 1210b and a third electrode layer 1210c (e.g., top electrode layer). A base layer 1212 between one of the electrode layers (e.g., bottom electrode layer) and the substrate can provide rigidity to the membrane during deflection. FIG. 12B shows a broad perspective view of the PMUT 1200 showing how multiple piezoelectric stacks 1202a, 1202b, 1202c, 1202d, 1202e, 1202f, 1202g and 1202h can be concentrically arranged, with each of the piezoelectric stacks 1202a-1202h including a piezoelectric layers 1208a, 1208b, electrode layers 1210a, 1210b, 1210c, and base layer 1212.

The example of FIGS. 12A and 12B show a PMUT device having two piezoelectric layers. However, the PMUT devices described herein can include any number of piezoelectric layers (e.g., 1, 2, 3, 4, 5, 6, or more layers). In some examples, the piezoelectric layers may have a maximum overall stack thickness 1220 of the stacked membrane, depending on the particular application and size requirements. In theory, stacking of the piezoelectric layers can lead to a linear increase in amplitude of the vibration in theory, however, in practice fabrication errors may reduce the absolute amplitude. For example, the stack number may be increased from two to approximately 62. The optimal number of piezoelectric layers can be determined, e.g., by analyzing and quantifying the losses with the deposition of each stack. In some examples, the minimum number of piezoelectric layers is between 2-10, to achieve desired performance. In some examples, the PMUT device includes a stack having two to four piezoelectric layers.

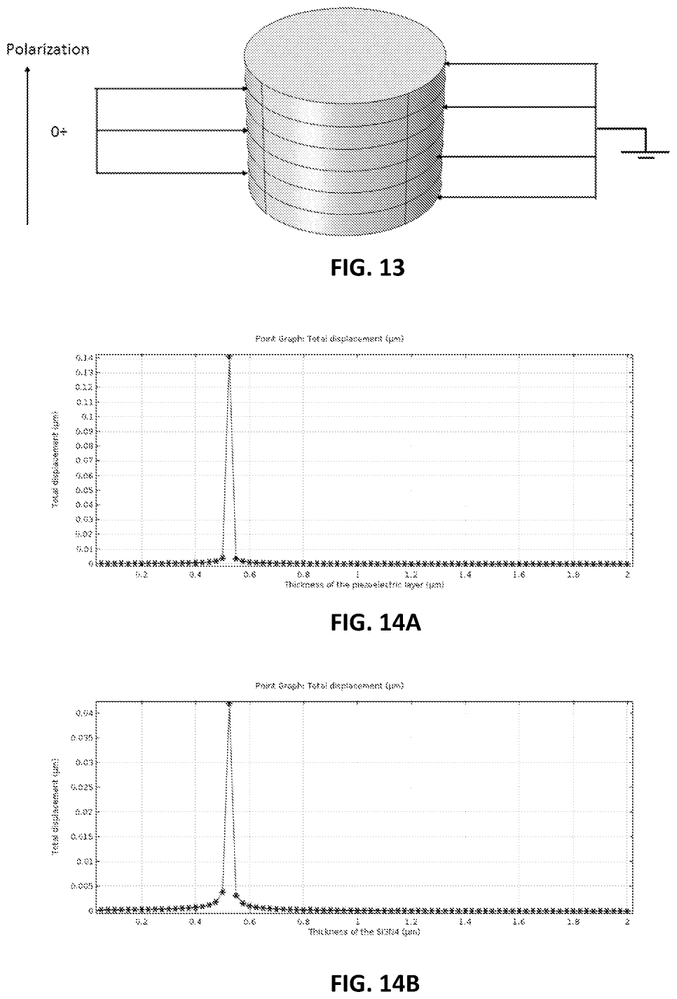

For PMUT devices having multiple piezoelectric layers, the piezoelectric layers may be arranged with alternating polarities. FIG. 13 shows an example of a stack of piezoelectric elements arranged in alternating polarity. When a voltage is applied parallel to the direction of polarization, a strain, or displacement, is induced in the direction of polarization. In FIG. 13, every other electrode layer (arrows) is coupled to a first electrical lead (e.g., electrode), and the electrodes between those are connected to a second electrical lead (e.g., shown as ground in FIG. 13). Thus, the electrode layers in the multilayered stack shown to alternate, with every other electrode layer (moving from the base layer up the height of the stack) are in electrical communication with each other (e.g., connected to a common lead or electrode) and the remaining electrodes are also in electrical communication with each other (e.g., connected to a second common lead or electrode, in this case ground). The movement of a piezoelectric element equals the amount of voltage applied multiplied by the piezoelectric coefficient, D₃₃, which relates to the material's efficiency in transferring electrical energy to mechanical energy. Because the piezoelectric elements are connected mechanically in series, the total movement of a stacked piezoelectric actuator is the product of a single element's movement times the number of elements in the stack (ΔL=n*D₃₃*V, where ΔL is the change of length in meters (m), n=number of piezo layers, V=operating voltage, and D₃₃=longitudinal piezo electric coefficient (m/V)). The total displacement of a stacked actuator may be between 0.1 and 0.15 percent of the stack height. By stacking multiple piezoelectric layers, a higher displacement for a given voltage can be obtained compared to a single piezoelectric layer. The alternating polarity allows the maintaining of a uniform electric field inside the stack and also allows ease of connection.

The materials of the various components of the PMUT devices may vary depending, in part, on performance requirements and other requirements related to the particular application. In some examples, the piezoelectric material of the piezoelectric layer includes one or more of a zinc oxide (ZnO), an aluminum nitride (AlN), an aluminum scandium nitride (AlScN), a lead magnesium niobate-lead titanate (PMN-PT) based material, and a polyvinylidene difluoride (PVDF) polymer. In some medical device applications, for instance, a lead-free device may be required and may not utilize a PMN-PT piezoelectric material. The thickness (also referred to as “height”) (e.g., 1107) of the piezoelectric layer(s) may vary depending, in part, on overall thickness requirements of the membrane (e.g., maximum thickness of the membrane) and the number of piezoelectric layers. In some examples where the device has a single piezoelectric layer (e.g., FIGS. 11A and 11B), the height of the piezoelectric layer may range from about 0.25 micrometers (μm) to about 3 μm (e.g., 0.25-3 μm, 0.5-2 μm, 0.5-1.5 μm, 0.75-1.25 μm, or 0.25-2 μm). The height of each piezoelectric layer in device that includes multiple piezoelectric layers may be less than the height of a piezoelectric layer having a single piezoelectric layer to avoid exceeding a maximum overall thickness of the membrane stack. In some examples where the device has multiple piezoelectric layers (e.g., FIGS. 12A and 12B), the height of each of the piezoelectric layers may range from about 0.1 (μm) to about 5 μm (e.g., about 0.1-4 μm, about 0.2-3 μm, about 0.25-2 μm, about 0.75-1.5 μm, about 0.5-2 μm, etc.). In some examples, the total height of the one or more piezoelectric layers (e.g., combined height if more than one piezoelectric layer) ranges from about 0.20 μm to about 5 μm (e.g., about 0.25-3 μm, about 0.5-2 μm, about 0.5-1.5 μm, about 0.75-1.25 μm, about 0.25-2 μm, etc.). As mentioned, these dimensions are only for illustration of particular examples; these dimensions may change based on the scale of the device and its intended frequency range.

In some examples, the base layer includes a piezo ceramic material. In some cases, the base layer includes silicon oxide (SiO₂) and/or silicon nitride (Si₃N₄). In some applications, a silicon nitride layer may be preferable as it may provide better responsiveness compared to silicon oxide for a given thickness.

The thickness of the base layer can depend, in part, on the thickness of the multilayer stack membrane. The base layer should be thick enough to provide sufficient rigidity to prevent the multilayer stack membrane from flexing too much and increasing the fragility of the device. However, the base layer should be thin enough to allow the multilayer stack membrane to sufficiently vibrate for piezoelectric functionality. For devices having a single piezoelectric layer (e.g., FIGS. 11A and 11B), the base layer thickness may range from about 200 nanometers (nm) to about 600 nm (e.g., about 200-600 nm, about 200-400 nm, about 300-400 nm, about 300-500 nm, etc.). For devices having two or more piezoelectric layers (e.g., FIGS. 12A and 12B), the base layer thickness may range from about 400 nm to about 700 nm (e.g., about 400-700 nm, about 400-600 nm, about 500-600 nm, about 500-700 nm, etc.). For devices having more than two piezoelectric layers, the base layer thickness may range, e.g., from about 500 nm to about 1000 nm (e.g., about 500-1000 nm, about 700-1000 nm, about 600-1000 nm, etc.). In some examples, for devices having two or more piezoelectric layers, the base layer may be at least 500 nm. These dimensions are for illustration only. As mentioned, the devices described herein may be scaled to larger or smaller dimensions based on the desired frequency characteristics and device use.

In some cases, the thickness of the piezoelectric layer(s) and the base layer are based on an eigenmode frequency of the device. FIGS. 14A and 14B are graphs showing results from 2D simulations based on a frequency mode of 76.75 MHz for a PMUT device. FIG. 14A shows a calculated total displacement of the membrane achieved for different thicknesses of the piezoelectric layer. These results indicate that a total piezoelectric layer(s) thickness between about 0.4 μm and 0.6 μm may provide optimal membrane displacement for a vibration frequency mode of 76.75 MHz. FIG. 14B shows a calculated total displacement of the membrane achieved for different thicknesses of the base layer. These results indicate that a base layer thickness between about 0.4 μm and 0.6 μm may provide optimal membrane displacement for a vibration frequency mode of 76.75 MHz.

The material of the electrode layers may vary depending on performance requirements and, in some cases, fabrication costs. In some examples, the electrode layers are made of or include aluminum, gold and/or platinum. In some examples, the electrode layers each include sublayers of different metals. For example, in some cases, each electrode layer includes a platinum layer and a titanium layer (e.g., 200:20 nm Pt/Ti), a platinum layer and a chromium layer (e.g., 200:20 nm Pt/Cr), a gold layer and a titanium layer (e.g., 200:20 nm Au/Ti), or a gold layer and a chromium layer (e.g., 200:20 nm Au/Cr). In some implementations, each electrode layer is made of different materials, which may provide good contrast in imaging applications. For instance, in some examples, a first electrode layer (e.g., bottom electrode layer) may be made of platinum and a second electrode layer (e.g., top electrode layer) may be made of gold. The thickness of the electrode layers may vary depending, in part, on the material(s) of the electrode layers. The electrode material should be thick enough to provide good adhesion and prevent acoustic losses, yet thin enough to avoid contributing too much to the overall thickness of the membrane. In some examples, each electrode layer can have a thickness ranging from about 100 nm to about 400 nm (e.g., 100-400 nm, 150-300 nm, 100-300 nm, or 200-400 nm).

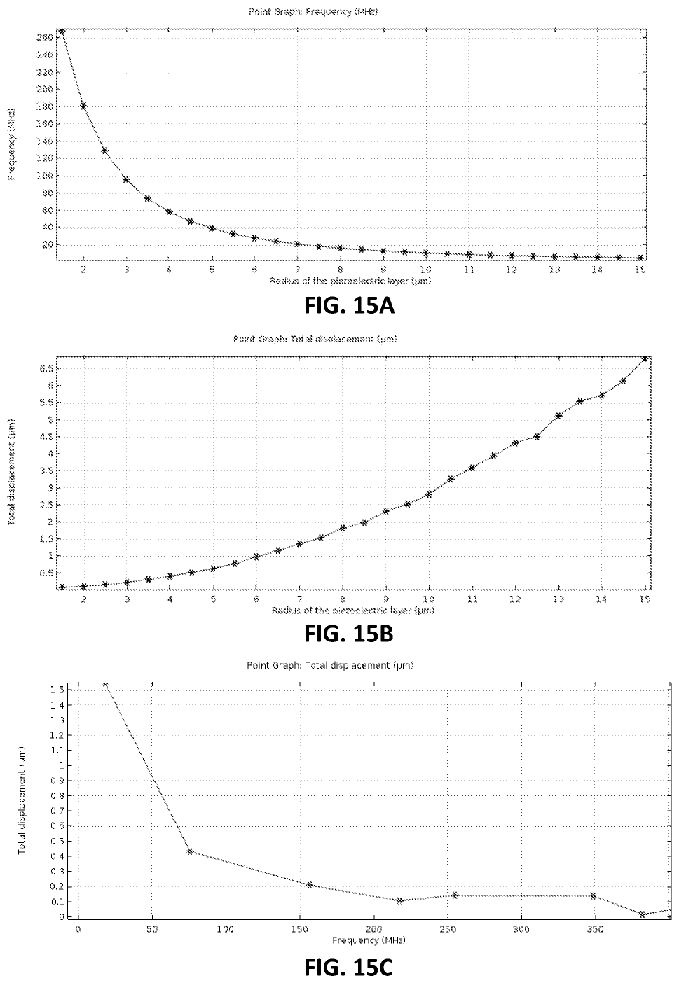

The dimensions of the various components of the PMUT devices may vary depending, in part, on performance requirements. FIGS. 15A-15C show results from a 2D simulation of a single PMUT cell to determine the effect of piezoelectric layer radius on vibration frequency and total displacement of a PMUT device. This information can be used estimate performance of a PMUT having a ring array arrangement. FIG. 15A is a plot showing the calculated variation of the principal mode frequency of a single PMUT cell with respect to the radius of the piezoelectric layer. FIG. 15B is a plot showing the calculated variation of total displacement of the principal mode frequency of a single PMUT cell with respect to the radius of the piezoelectric layer. The results indicate that the smaller the radius of the diaphragm of the device, the higher the principal mode frequency and the lower the vibration amplitude. FIG. 15C is a plot showing the calculated variation of the total displacement of the eigenmodes of a single PMUT cell have a particular cavity size (cavity radius of 15 μm with 40 μm by 40 μm piezoelectric cell dimensions) at different vibration frequencies. Note that even though the PMUT of FIG. 15C (having a cavity radius of 15 μm and 40 μm by 40 μm cell dimensions) is calculated to have a principal mode frequency of 14.4 MHz, the PMUT can be excited at a higher frequency mode (e.g., about 76.67 MHz) with a lower vibration amplitude. In some applications, a ring array PMUT device having piezoelectric stacks with the following dimensions are found to provide performance well suited for catheter sensing applications: a cavity height (e.g., FIG. 11A, 1116) and piezoelectric layer radius/width (e.g., FIG. 11A, 1119) ranging from about 50 μm to about 300 μm (e.g., 50-300 μm, 100-200 μm, or 75-150 μm), a cavity radius/width (e.g., FIG. 11A, 1118) ranging from about 5 μm to about 20 μm (e.g., 5-20 μm, 10-20 μm, or 15-30 μm).

The number of piezoelectric stack rings and the ring pitch (distance between the rings) may also be selected based on simulations (e.g., 2D and/or 3D simulations) of a single PMUT cell. The example PMUT devices of FIGS. 11A-11B and 12A-12B include eight concentrically arranged piezoelectric stacks, however, the PMUT devices may include any number of concentrically arranged piezoelectric stacks (e.g., 1, 2, 3, 4, 5, 6, 7, 8, 9, 10, 11 or more). In some examples, the ring pitch ranges from about 0.25 μm to about 3 μm (e.g., 0.25-3 μm, 1-2 μm, 0.5-1.5 μm, or 1-2 μm). In one implementation, a ring pitch of 1 μm is maintained to allow eight stacks for a 30 μm PMUT cell. This can improve the tunability by enforcing the excitation frequency of the PMUT cell.

The overall dimensions of the PMUT device may be small so that one or more of the PMUT devices may be integrated within a small medical device, such as an ultrasound imaging catheter. For example, in some examples, as shown in FIG. 22A an array of PMUTs may be arranged on a device. In FIG. 22A, a linear array 2201 of PMUT cells 2205 as described herein are shown. In this example an array of PMUTs as described herein form a line. In some examples the PMUT cells 2205 may be arranged on a device (e.g., as part of a catheter, for example) to form a side-facing array. For example, a line arrangement of a PMUT cells may be placed around a catheter with each line array (e.g., having an angular aperture of, for example,) 5.625° forming a side-viewing ring array. Other array configurations are possible, including side-viewing linear arrays, forward-viewing ring arrays, forward-viewing linear arrays, etc.). FIG. 22B illustrates an arrangement of PMUTs 2205 configured as a side-viewing ring array 2203 around the catheter. FIG. 22C illustrates an arrangement of PMUTs 2205 configured as a side-viewing helical/spiral array 2207.

In another example, a ring array having a diameter from 5 μm to 35 μm, for a minimum of 64 elements, may be positions on a 3 Fr catheter. Larger catheters may use larger ring arrays.

Another performance parameter of the PMUT device is penetration depth, which corresponds to the minimum scan depth at which electronic noise is visible, despite optimization of available controls (usually at the deepest transmit focal setting and maximum gain), and electronic noise stays at a fixed depth even when the PMUT is moved laterally. Penetration can primarily be determined by the center frequency of the transducer: the higher the frequency, the shallower the penetration because the absorption of the ultrasound wave traveling through tissue increases with frequency. A useful first approximation for estimating a depth of penetration (dp) for a given frequency is dp=60/f cm-MHz, where f is in MHz. The absorption coefficient (acoustic power loss per unit depth) is a function of frequency and varies from tissue to tissue (values for soft tissues range from 0.6 to 1.0 dB/cm-MHz). A more general term describing acoustic loss is the attenuation coefficient, which includes additional losses due to scattering and diffusion and hence is always greater than the absorption coefficient. The attenuation coefficient is highly patient and acoustic path dependent, hence it is difficult to simulate accurately. In order to have a simulation model that predicts it accurately, values can be extracted from experimental data and added to the models to obtain a robust model. The following are example PMUT devices.

### Example 1: Single Piezoelectric Layer PMUT Device

Simulations were performed based on a PMUT device having a ring array configuration with piezoelectric stacks having a single piezoelectric layer (FIGS. 11A and 11B) according to the specification in Table 1 below.

TABLE 1

| Number of piezoelectric layers | 1 | |
|---|---|---|
| Piezoelectric layer height | 1 | μm |
| Piezoelectric layer radius | 15 | μm |
| Base layer material | Si₃N₄ | |
| Base layer thickness | 300 | nm |
| Cell width | 40 μm × 40 μm | |
| Cavity radius | 15 | μm |
| Cavity height | 125 | μm |
| Electrode layers material | Al | |
| Electrode layer thickness (each) | 200 | nm |
| Ring pitch | 1 | μm |

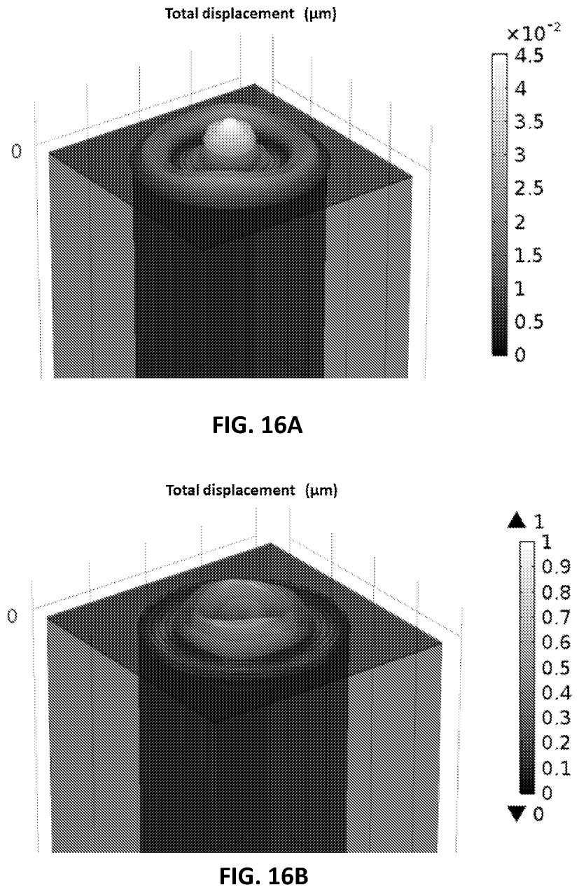

FIG. 16A shows results from a 3D simulation model based on calculated resonant modes and displacement fields for the PMUT with a single zinc oxide (ZnO) piezoelectric layer. A working frequency of 63.63 MHz and a total displacement of 50 nanometers (nm) is calculated using the ring PMUT with a ZnO piezoelectric layer, which is approximately a ten times greater in amplitude compared to a circular PMUT with a ZnO piezoelectric layer. FIG. 16B shows results from a 3D simulation model based on calculated resonant modes and displacement fields for a ring array PMUT with a single aluminum nitride (AlN) piezoelectric layer. A working frequency of 63.59 MHz and a total displacement of 1 micrometer (μm) is calculated using the ring PMUT with an AlN piezoelectric layer, which is approximately a 71 times greater in amplitude compared to a circular PMUT with an AlN piezoelectric layer.

### Example 2: Double Piezoelectric Layer PMUT Device

Simulations were performed based on a PMUT device having a ring array configuration with piezoelectric stacks having two piezoelectric layers (FIGS. 12A and 12B) according to the specification in Table 2 below.

TABLE 2

| Number of piezoelectric layers | 2 | |
|---|---|---|
| Piezoelectric layer height (each) | 0.5 | μm |
| Piezoelectric layer radius | 15 | μm |
| Piezoelectric layer material | ZnO | |
| Base layer material | Si₃N₄ | |
| Base layer thickness | 500 | nm |
| Cell width | 40 μm × 40 μm | |
| Cavity radius | 15 | μm |
| Cavity height | 125 | μm |
| Electrode layers material | Al | |
| Electrode layer thickness (each) | 200 | nm |
| Overall stack thickness | 1600 | nm |
| Ring pitch | 1 | μm |

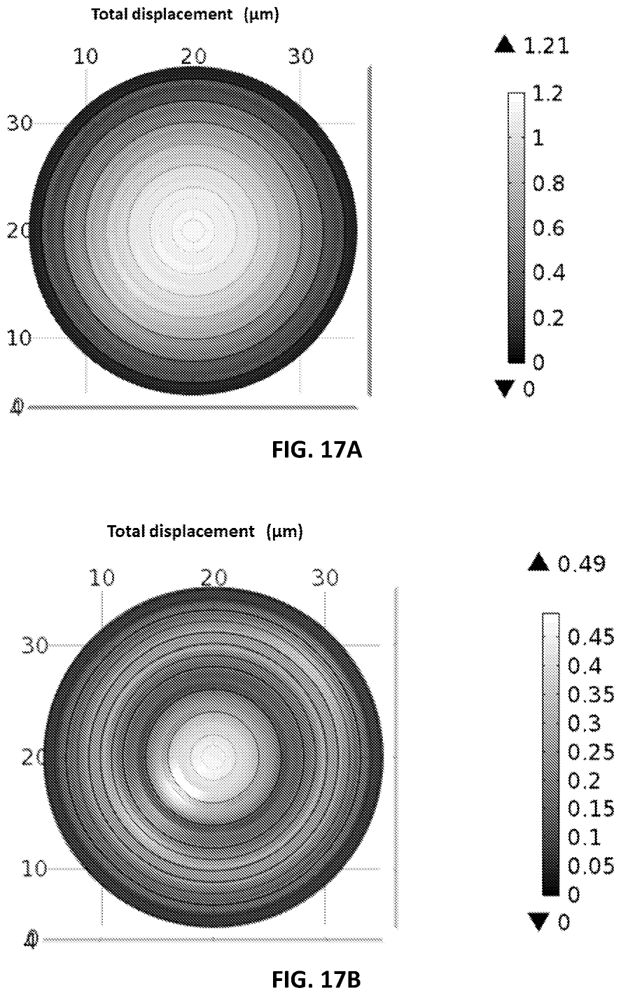

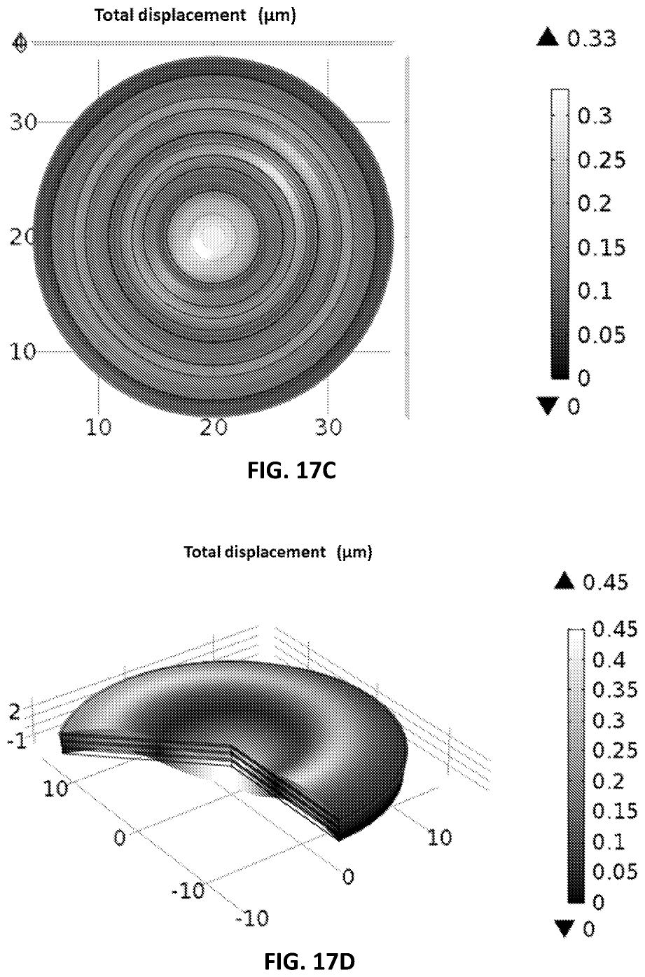

FIGS. 17A-17D illustrate simulation results showing total displacement (μm) of the ring array PMUT for three working resonant frequencies: 13.54 MHz (FIG. 17A), 42.92 MHz (FIG. 17B) and 78.98 MHz (FIGS. 17C and 17D). The calculated penetration depth of the PMUT device for three working resonant frequencies are summarized in Table 3 below.

TABLE 3

| Frequency | Calculated penetration |
|---|---|
| (MHz) | depth (60/frequency) |
| 13.54 | 4.43 cm-MHz |
| 42.92 | 1.39 cm-MHz |
| 78.98 | 0.75 cm-MHz |

The results indicate that the PMUT device can have a working frequency between about 10 MHz and 20 MHz and a penetration depth of at least 4 cm; a working frequency between about 70 MHz and 80 MHz and a penetration depth of at least 0.6 cm (e.g., greater than 0.6 cm); and/or a working frequency between about 35 MHz and 45 MHz and a penetration depth of at least 1 cm. Even the highest working frequency of the PMUT (around 70-80 MHz) provides a high penetration depth for a small device (e.g., for a 3 French catheter).

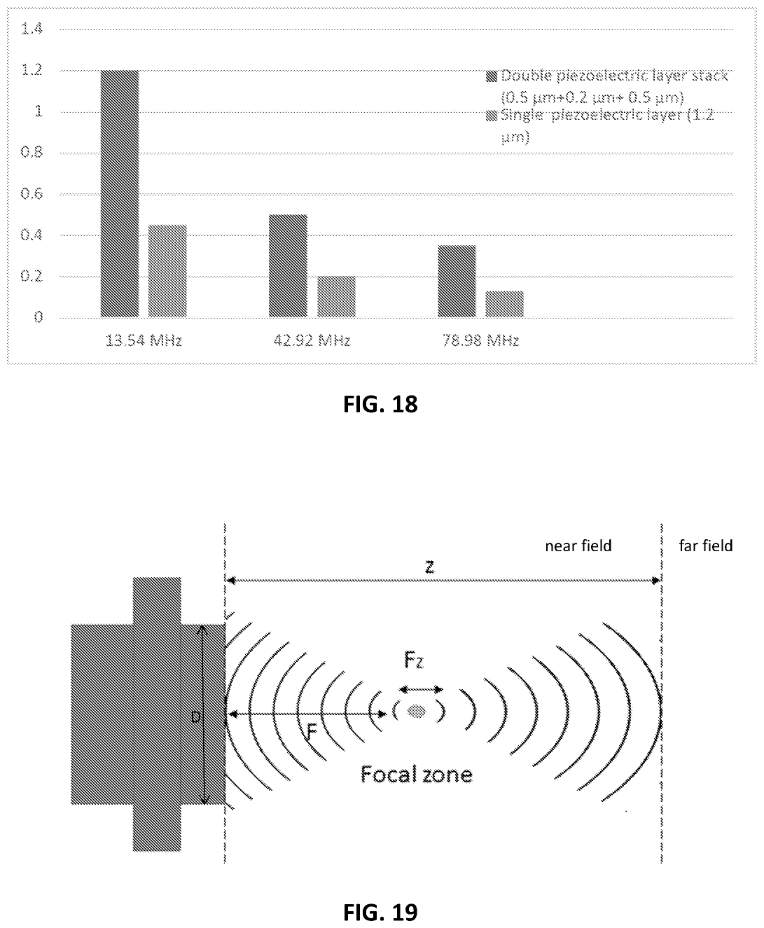

FIG. 18 is a graph comparing simulation results for calculated total displacement (μm) for resonant modes of a ring array PMUT having two ZnO piezoelectric layers and a ring array PMUT having a single ZnO piezoelectric layer of the same thickness. The simulation results indicate that the ring array PMUT having two ZnO piezoelectric layers provides a gain in frequency that provides higher resolution and the doubling of the piezoelectric stack provides an increase in total displacement with unit driving voltage compared to a single piezoelectric stack of the same thickness.

In some applications, the PMUT devices are implemented in ultrasound imaging catheters. The high penetration depth and small size of the piezoelectric transducers described herein can make the transducers well suited for integrating into/onto the small diameter catheters. In general, the focal length of a transducer is the distance from the face of the transducer to the point in the sound field where the signal with the maximum amplitude is located. In an unfocused transducer, this occurs at a distance from the face of the transducer which is approximately equivalent to the transducer's near field length. FIG. 19 shows a schematic representation of an acoustic field generated by an ultrasound transducer. Because the last signal maximum occurs at a distance equivalent to the near field, a transducer cannot be acoustically focused at a distance greater than its near field. The near field distance N (shown as “Z” in FIG. 19) is calculated as:

N=D²f/4c

where D is the transducer diameter, f is the frequency, and c is the speed of sound in the medium (in blood, 1540 m/s). The focal distance F is the distance between the transducer and the focal point that is the target zone. Individual PMUT cells (e.g., simple round or rectangular shaped cells) may not be focused, hence their focal length can be considered to be the length of their near field. PMUT cells assembled in an array (e.g., as a ring array of stacks), such as the ring arrays described herein, can produce the focusing effect. The calculated near field distance N for the PMUT having two piezoelectric layers (Example 2) at different resonant modes of interest are provided in Table 4 below.

TABLE 4

| Frequency | Near field distance (N) | |
|---|---|---|
| (MHz) | of a single cell | |
| 13.54 | 1.9782 | μm |
| 42.92 | 6.2708 | μm |
| 78.98 | 11.5397 | μm |

The calculated results indicate that the PMUT device can have a working frequency between about 10 MHz and 20 MHz and a near field distance of about 1 μm to about 3 μm; a working frequency between about 70 MHz and 80 MHz and a near field distance of about 5.5 μm to about 6.5 μm; and/or a working frequency between about 35 MHz and 45 MHz and a near field distance of about 10.5 μm to about 12.5 μm.

One or more of the ring array PMUT devices can be incorporated in and/or on the catheters. In some cases, the one or more transducers form a circular ring around the catheter. In some cases, one or more transducers are on the exterior walls of the imaging catheter, for example, at or near a distal end of the catheter. In such an arrangement, the transducer(s) may capture images along the side of the catheter (e.g., radially outward from a central axis of the catheter) to provide a side view along the catheter. Alternatively or additionally, the one or more transducers may be positioned at the distal tip of the imaging catheter. In such an arrangement, the transducer(s) may be configured to capture images from the front (e.g., distal tip) of the catheter for a forward view from the catheter. In some cases, the transducers for use in medical imaging can be lead-free. Thus, for example, the piezoelectric material may be made non-lead-based materials, such as zinc oxide.

The PMUT devices described herein can include those having any number of shapes and arrangements, and are not limited to the examples of FIGS. 11A-11B and 12A-12B. In some examples, the piezoelectric cells may include concentrically arranged polygonal (e.g., square, triangular, rectangular, pentagonal or hexagonal), elliptical or oval shaped rings rather than circular-shaped rings. In some examples, one or more piezoelectric stacks may have spiral/helical shape that winds from the center of the transducer. Different shapes and configurations may provide certain directionalities to the transducers, which may be useful in certain applications. However, certain shapes may increase the complexity of the design and fabrication of the devices. Thus, simpler configurations may be desirable and may provide suitable performance for certain applications.

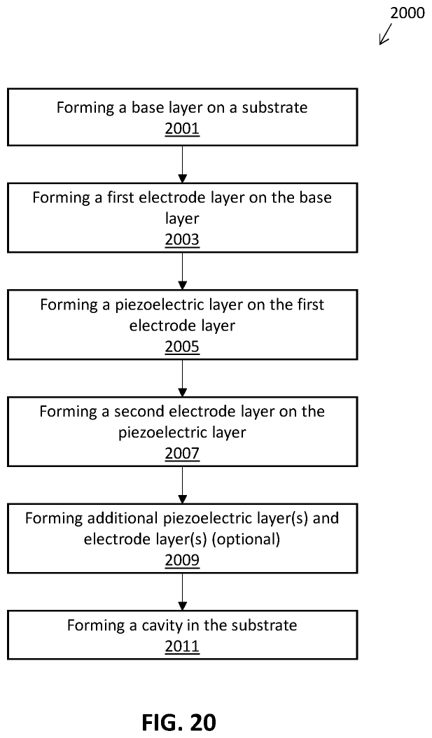

FIG. 20 shows a flowchart 2000 indicating a method of forming piezoelectric cells of a PMUT device according to some examples. A variety of different fabrication techniques may be use. In one example, to form the piezoelectric cells in accordance with a ring array structure, each of the processes 2001-2011 can be performed on the substrate (e.g., wafer) in accordance with the ring array pattern (e.g., FIG. 11B or 12B). At 2001, a base layer can be formed on the substrate. In some cases, the base layer is formed by a deposition process, such as plasma-enhanced chemical vapor deposition (PECVD) or low pressure chemical vapor deposition (LPCVD). In some cases, the substrate (e.g., silicon) is cleaned or etched prior to deposition. For example, a thermal oxide may be removed using an acidic solution (e.g., hydrofluoric acid). In some cases, a base layer is deposition on both sides—to be used as the mask for the substrate (e.g., silicon) anisotropic wet etching. At 2003, a first (e.g., bottom) electrode layer is formed on the base layer. In some examples, the base layer is deposited by ion-beam sputtering, patterned by photolithographic techniques, and wet etched (e.g., using H₃PO₄ solution). Alternatively or additionally, the multi-layer stack may be deposited on the substrate (e.g., all the layers) and the patterns may be etched for the individual electrodes and the piezoelectric layers one at a time.

At 2005, a piezoelectric layer is formed on the first electrode layer. In some examples, the piezoelectric layer is formed using a sputtering process, such as a magnetron sputtering process. In some cases, the piezoelectric layer is deposited to achieve a crystalline structure conducive with providing good piezoelectric properties. For example, a ZnO layer may exhibit a densely packed structure with columnar crystallites preferentially orientated along the (002) plane. In some cases, the piezoelectric layer is patterned (e.g., by wet etching using H₃PO₄ solution). At 2007, a second (e.g., top) electrode layer is formed on the piezoelectric layer. In some examples, the second electrode layer is deposited by ion-beam sputtering and photolithographically patterned by lift-off processing. At 2009, the method may optionally involve forming one or more additional piezoelectric layer and electrode layers to form a PMUT having multiple piezoelectric layers. This can involve repeating processes 2005 and 2007.

At 2011, a cavity is formed in the substrate. In some cases, forming the cavity involves a number of processes. In some cases, forming the cavity involves a back side film (e.g., Au/Cr) deposition, where the back side film is deposited on the back side of the wafer and patterned by back-to-front alignment photolithography techniques and a wet etching process, followed by inductively coupled plasma dry etching of based layer to form the mask for substrate (e.g., silicon) wet etching. In some cases, forming the cavity involves a back side mask etching process, where the wafer substrate is anisotropically etched using an etchant (e.g., KOH etchant at 70 C) to release the diaphragm. In some cases, forming the cavity involves a bulk machining process, where the bulk substrate material (e.g., silicon) is etched (e.g., wet etched) until the required cavity thickness is achieved. In some cases, forming the cavity involves a back side oxide etching, where oxide is removed by acidic solution (e.g., hydrofluoric acid solution) from the diaphragm. In some cases, fabrication of the PMUT device includes a wafer washing process, where the wafer is washed (e.g., with deionized water) after unloading from a fixture.

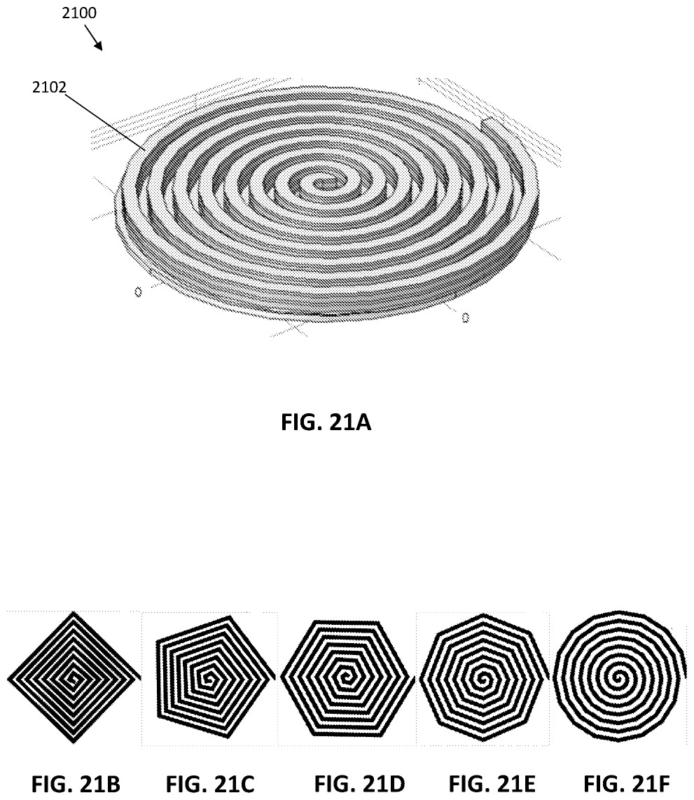

As mentioned above, the apparatuses described herein generally include a plurality of concentrically arranged multilayered stacks. In some examples (e.g., shown in FIGS. 11B and 12B) the concentrically arranged multilayered stacks are formed from a plurality of separate ring-shaped, concentric, multilayered stacks. Alternatively, in some examples the concentrically arranged multilayered stacks may be formed from a continuous spiral, as shown in FIGS. 21A and 21B-21F. For example, in FIG. 21A, the cell is formed of a single spiral forming the concentrically arranged multilayered stacks 2102. The spiral wraps outward with a constant pitch (center to center distance between 2 electrodes), constant electrode thickness and width. Thus, the spiral shown in FIG. 21A has eight loops that are concentrically arranged (and continuous with each other).

Similarly, FIGS. 21B-21F show other examples of ultrasound transducer apparatuses (e.g., PMUT devices) as described herein, in which the plurality of concentrically arranged multilayered stacks on the substrate are formed from a polygonal spiral. FIGS. 21A-21F all show different polygonal shapes of the PMUT cell where the number of sides of the PMUT cell in 4 in FIG. 21B, 5 in FIG. 21C, 6 in FIG. 21D, 8 in FIG. 21E, and 16 in FIG. 21F. Thus, a plurality of concentrically arranged multilayered stacks may refer to two or more separate and concentrically arranged stacks, a single concentrically spiraling multilayered stack, multiple concentrically spirally multilayered stacks or a combination of a concentrically spirally stack and one or more separate encircled/encircling stacks.

In any of the apparatuses described herein the plurality of concentrically arranged multilayered stacks may be separated by a gap (e.g., an air gap or space) between the concentrically arranged stacks. In FIGS. 21A-21F the gap is approximately the same width as the stack; in some examples the gap may be larger or smaller.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one example, the features and elements so described or shown can apply to other examples. It will also be appreciated by those of skill in the art that references to a structure or feature that is disposed “adjacent” another feature may have portions that overlap or underlie the adjacent feature.

Terminology used herein is for the purpose of describing particular examples only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative examples are described above, any of a number of changes may be made to various examples without departing from the scope of the invention as described by the claims. For example, the order in which various described method steps are performed may often be changed in alternative examples, and in other alternative examples one or more method steps may be skipped altogether. Optional features of various device and system examples may be included in some examples and not in others. Therefore, the foregoing description is provided primarily for exemplary purposes and should not be interpreted to limit the scope of the invention as it is set forth in the claims.

The examples and illustrations included herein show, by way of illustration and not of limitation, specific examples in which the subject matter may be practiced. As mentioned, other examples may be utilized and derived there from, such that structural and logical substitutions and changes may be made without departing from the scope of this disclosure. Such examples of the inventive subject matter may be referred to herein individually or collectively by the term “invention” merely for convenience and without intending to voluntarily limit the scope of this application to any single invention or inventive concept, if more than one is, in fact, disclosed. Thus, although specific examples have been illustrated and described herein, any arrangement calculated to achieve the same purpose may be substituted for the specific examples shown. This disclosure is intended to cover any and all adaptations or variations of various examples. Combinations of the above examples, and other examples not specifically described herein, will be apparent to those of skill in the art upon reviewing the above description.

It will be understood to persons skilled in the art of the invention that many modifications may be made without departing from the spirit and scope of the invention.

In the claims which follow and in the preceding description of the invention, except where the context requires otherwise due to express language or necessary implication, the word “comprise” or variations such as “comprises” or “comprising” is used in an inclusive sense, i.e., to specify the presence of the stated features but not to preclude the presence or addition of further features in various examples of the invention.

It is to be understood that, if any prior art publication is referred to herein, such reference does not constitute an admission that the publication forms a part of the common general knowledge in the art, in Australia or any other country.

What is claimed is:

1. An imaging balloon catheter device, the device comprising:
an inflatable balloon element;
an elongate tubular body having a distal end and a proximal end, the distal end for insertion into a vessel and the proximal end configured to remain external to the vessel accessible to a clinician and connectable to external components, the distal end being configured to support the inflatable balloon element and enable inflation and deflation of the inflatable balloon element; and
an imaging sensor assembly on the elongate tubular body at a distal location relative to the inflatable balloon element, the imaging sensor assembly including an array of piezoelectric micromachined ultrasound transducer (PMUT) sensors arranged in a ring or helix around the elongate tubular body, the imaging sensor assembly being in data communication with an image processing system configured to process data output from the imaging sensor assembly and render for display.

2. The device of claim 1, wherein the imaging sensor assembly is configured to enable 360 degree imaging of the vessel interior.

3. The device of any of claims 1-2, wherein the elongate tubular body supports one or more electrical conductors to provide power and data connection to the imaging sensor assembly.

4. The device of claim 3, wherein the electrical conductors are wires connecting each of the PMUT sensors in the array to a bus extending longitudinally along an internal lumen wall of the elongate tubular body.

5. The device of any of claims 1-4, wherein the imaging sensor assembly is further configured for any one or more of electromagnetic induction tomography, electrical capacitance tomography, electrical resistance tomography, electrical induction tomography, near infrared spectroscopy, optical coherence tomography, photography or videography.

6. The device of any of claims 1-5, further comprising a processor configured to analyze and display image data.

7. The device of any of claims 1-6, comprising a processor configured to monitor movement of the imaging balloon catheter device as the imaging balloon catheter device is extracted, and wherein movement data is input to analysis of sampled image data.

8. The device of claim 7, further comprising a mechanism for controlling extraction of the imaging balloon catheter device, whereby the imaging balloon catheter device can be extracted at a pre-set pull back rate.

9. The device of claim 7, wherein monitoring movement of the imaging balloon catheter device is based on marker detection via an angiogram feed or via markers on the imaging balloon catheter device external to the vessel and visually accessible to a clinician.

10. The device of claim 7, further comprising one or more motion sensors mounted on the elongate tubular body to enable monitoring of movement of the imaging balloon catheter device.

11. The device of claim 10, wherein the one or more motion sensors comprise one or more of an accelerometer, a gyroscope, a Global Positioning System (GPS) sensor, a velocity sensor, a position sensor, and an optical sensor.

12. The device of any of claims 1-11, wherein an outer diameter of the imaging balloon catheter device is of 3 French or less.

13. The device of any of claims 1-12, wherein each of the PMUT sensors of the array includes a multilayered stack including a plurality of piezoelectric layers arranged between electrode layers, wherein the multilayered stack is arranged over a cavity of a substrate.

14. The device of claim 1-12, wherein each PMUT sensor of the array comprises a plurality of concentric multilayered stacks extending proud of a base layer, the plurality of concentric multilayered stacks and base layer arranged over a cavity, wherein the concentric multilayered stacks are separated by a space, further wherein each of the concentric multilayered stacks includes a plurality of piezoelectric layers, and wherein each piezoelectric layer is arranged between electrode layers.

15. The device of any of claims 13-14, wherein the base layer has a thickness of at least 500 nanometers.

16. The device of any of claims 13-14, wherein the piezoelectric layers alternate in polarity along a direction of a height of the stack.

17. The device of any of claims 13-14, wherein each of the stacks includes between two and eight piezoelectric layers.

18. The device of any of claims 13-14, wherein the piezoelectric layers each have a thickness ranging from 0.25 micrometers to 3 micrometers.

19. The device of any of claims 1-18, wherein the imaging sensor assembly has a working frequency between 70 MHz and 80 MHz and has a penetration depth of at least 0.6 cm.

20. The device of any of claims 1-18, wherein the imaging sensor assembly has a working frequency between 35 MHz and 45 MHz and has a penetration depth of at least 1 cm.

21. The device of any of claims 1-18, wherein the imaging sensor assembly has a working frequency between 10 MHz and 20 MHz and has a penetration depth of at least 4 cm.

22. An imaging balloon catheter device, the device comprising:
an inflatable balloon element;
an elongate tubular body having a distal end and a proximal end, the distal end for insertion into a vessel and the proximal end configured to remain external to the vessel accessible to a clinician and connectable to external components, the distal end being configured to support the inflatable balloon element and enable inflation and deflation of the inflatable balloon element; and
an imaging sensor assembly on the elongate tubular body at a distal location relative to the inflatable balloon element, the imaging sensor assembly including an array of piezoelectric micromachined ultrasound transducer (PMUT) sensors on the elongate tubular body, wherein each of the PMUT sensors of the array includes a multilayered stack including a plurality of piezoelectric layers arranged between electrode layers, wherein the multilayered stack is arranged over a cavity of a substrate.

23. A method of imaging within a vessel during a catheterization procedure, the method comprising:
positioning a balloon catheter within a vessel, the balloon catheter including an imaging sensor assembly on an elongate tubular body, the imaging sensor assembly distally located relative to an inflatable balloon element, the imaging sensor assembly including piezoelectric micromachined ultrasound transducer (PMUT) sensors arranged around the elongate tubular body in a ring or helix;
receiving an input triggering start of imaging; and
in response to receiving the input:
monitoring movement of the balloon catheter to provide movement data characterizing movement of the balloon catheter;
sampling image data using the imaging sensor assembly;
processing the sampled image data using the movement data to render images of the vessel; and
display the sampled image data.

24. The method of claim 23, further comprising determining a treatment area length based on the sampled image data.

25. The method of any of claims 23-24, wherein the treatment area length is determined based on identification of a start and end of a vessel wall abnormality based on the sampled image data and calculation of a distance between the start and end.

26. The method of any of claims 23-25, further comprising determining a location of the balloon catheter using a radio opaque marker.

27. The method of any of claims 23-26, further comprising rotating the balloon catheter during imaging.

28. The method of any of claims 23-27, wherein sampling image data using the imaging sensor assembly comprises applying a voltage between a plurality of electrode layers in each PMUT sensor, wherein each PMUT comprises a plurality of concentric multilayered stacks, each multilayered stack extending proud of a base layer over a cavity, further wherein each of the concentric multilayered stacks includes a plurality of piezoelectric layers, and wherein each piezoelectric layer is arranged between two electrode layers of the plurality of electrode layers; and inducing, from the applied voltage, a displacement that is a proportional to the applied voltage, a piezoelectric coefficient of a material forming the piezoelectric layers, and the number of piezoelectric layers.

29. The method of claim 28, wherein inducing the displacement comprises inducing a displacement at a frequency of between about 70 MHz and 80 MHz, wherein a penetration depth of an ultrasound signal emitted by the displacement is at least 0.6 cm.

30. The method of claim 28, wherein inducing the displacement comprises inducing a displacement at a frequency of between about 35 MHz and 45 MHz, wherein a penetration depth of an ultrasound signal emitted by the displacement is at least 1 cm.

31. The method of claim 28, wherein inducing the displacement comprises inducing a displacement at a frequency of between about 10 MHz and 20 MHz, wherein a penetration depth of an ultrasound signal emitted by the displacement is at least 4 cm.

32. A method of imaging within a vessel during a catheterization procedure, the method comprising:
positioning a balloon catheter within a vessel, the balloon catheter including an imaging sensor assembly on an elongate tubular body, the imaging sensor assembly distally located relative to an inflatable balloon element, the imaging sensor assembly including piezoelectric micromachined ultrasound transducer (PMUT) sensors;
receiving an input triggering start of imaging; and
in response to receiving the input:
monitoring movement of the balloon catheter to provide movement data characterizing movement of the balloon catheter;
sampling image data using the imaging sensor assembly by applying a voltage between a plurality of electrode layers in each PMUT sensor, wherein each PMUT comprises a plurality of concentric multilayered stacks, each multilayered stack extending proud of a base layer over a cavity, further wherein each of the concentric multilayered stacks includes a plurality of piezoelectric layers, and wherein each piezoelectric layer is arranged between two electrode layers of the plurality of electrode layers; and inducing, from the applied voltage, a displacement that is a proportional to the applied voltage, a piezoelectric coefficient of a material forming the piezoelectric layers, and the number of piezoelectric layers;
processing the sampled image data using the movement data to render images of the vessel; and
display the sampled image data.

33. A combined imaging balloon catheter device, the device comprising:
a catheter having a distal end with a flexible tip having an outer diameter of 3 French or less;
an inflatable balloon assembly on the catheter proximal to the flexible tip; and
an imaging assembly in the flexible tip distal to the inflatable balloon assembly, the imaging assembly including an array of 4 or more piezoelectric micromachined ultrasound transducer (PMUT) sensors arranged in a ring or helix;
wherein the flexible tip has a length and flexibility sufficient to house the imaging assembly for imaging a portion of a blood vessel or lumen to be dilated or stented without puncturing a wall of the blood vessel or lumen.

34. The device of claim 33, wherein the inflatable balloon assembly comprises a compliant or non-compliant balloon.

35. The device of any of claims 33-34, wherein the flexible tip narrows distally away from one or both of the inflatable balloon assembly and the imaging assembly.

36. The device of any of claims 33-35, wherein each PMUT sensor of the array comprises a plurality of concentric multilayered stacks extending proud of a base layer, the plurality of concentric multilayered stacks and base layer arranged over a cavity, wherein the concentric multilayered stacks are separated by a space, further wherein each of the concentric multilayered stacks includes a plurality of piezoelectric layers, and wherein each piezoelectric layer is arranged between electrode layers.

37. The device of claim 36, wherein the piezoelectric layers each have a thickness ranging from 0.25 micrometers to 3 micrometers.

38. The device of claim 36, wherein the array of PMUT sensors has a working frequency between 70 MHz and 80 MHz and has a penetration depth of at least 0.6 cm.

39. The device of claim 36, wherein the array of PMUT sensors has a working frequency between 35 MHz and 45 MHz and has a penetration depth of at least 1 cm.

40. The device of claim 36, wherein the array of PMUT sensors has a working frequency between 10 MHz and 20 MHz and has a penetration depth of at least 4 cm.

41. A combined imaging balloon catheter device, the device comprising:
a catheter having a distal end with a flexible tip having an outer diameter of 3 French or less;
an inflatable balloon assembly on the catheter proximal to the flexible tip; and
an imaging assembly in the flexible tip distal to the inflatable balloon assembly, the imaging assembly including an array of piezoelectric micromachined ultrasound transducer (PMUT) sensors, wherein each PMUT sensor of the array comprises a plurality of concentric multilayered stacks extending proud of a base layer, the plurality of concentric multilayered stacks and base layer arranged over a cavity, wherein the concentric multilayered stacks are separated by a space, further wherein each of the concentric multilayered stacks includes a plurality of piezoelectric layers, and wherein each piezoelectric layer is arranged between electrode layers;
wherein the flexible tip has a length and flexibility sufficient to house the imaging assembly for imaging a portion of a blood vessel or lumen to be dilated or stented without puncturing a wall of the blood vessel or lumen.

42. A method of sizing a stent for a blood vessel or lumen, the method comprising:
positioning a combined imaging balloon catheter within the blood vessel or lumen, the combined imaging balloon catheter including:
a catheter having a distal end with a flexible tip having an outer diameter of 3 French or less;
an inflatable balloon assembly on the catheter proximal to the flexible tip; and
an imaging assembly in the flexible tip distal to the inflatable balloon assembly, the imaging assembly including an array of 4 or more piezoelectric micromachined ultrasound transducer (PMUT) sensors arranged in a ring or helix;
imaging a portion of the blood vessel or lumen to be stented using the imaging assembly of the combined imaging balloon catheter without the inflatable balloon assembly being passed wholly through the portion of the blood vessel or wholly pulled back through the portion of the blood vessel or lumen; and
determining a stent size based at least in part on the imaging of the portion of the blood vessel or lumen.

43. The method of claim 42, wherein determining the stent size comprises determining a treatment area length based on collected image data.

44. The method of claim 43, wherein the treatment area length is determined based on identification of a start and end of a vessel wall abnormality based on the collected image data and a calculation of a distance between the start and end.

45. The method of any of claims 42-44, further comprising measuring longitudinal and rotational movement of the combined imaging balloon catheter within the blood vessel or lumen.

46. The method of any of claims 42-45, wherein the inflatable balloon assembly comprises a compliant or non-compliant balloon.

47. The method of any of claims 42-46, wherein the flexible tip narrows distally away from one or both of the inflatable balloon assembly and the imaging assembly.

48. The method of any of claims 42-47, wherein imaging the portion of the blood vessel or lumen to be stented comprises applying a voltage between a plurality of electrode layers in each PMUT sensor of the array of PMUT sensors, wherein each PMUT sensor comprises a plurality of concentric multilayered stacks, each multilayered stack extending proud of a base layer over a cavity, further wherein each of the concentric multilayered stacks includes a plurality of piezoelectric layers, and wherein each piezoelectric layer is arranged between two electrode layers of the plurality of electrode layers; and inducing, from the applied voltage, a displacement that is a proportional to the applied voltage, a piezoelectric coefficient of a material forming the piezoelectric layers, and the number of piezoelectric layers.

49. The method of claim 48, wherein inducing the displacement comprises inducing a displacement at a frequency of between about 70 MHz and 80 MHz, wherein a penetration depth of an ultrasound signal emitted by the displacement is at least 0.6 cm.

50. The method of claim 48, wherein inducing the displacement comprises inducing a displacement at a frequency of between about 35 MHz and 45 MHz, wherein a penetration depth of an ultrasound signal emitted by the displacement is at least 1 cm.

51. The method of claim 48, wherein inducing the displacement comprises inducing a displacement at a frequency of between about 10 MHz and 20 MHz, wherein a penetration depth of an ultrasound signal emitted by the displacement is at least 4 cm.

52. A method of sizing a stent for a blood vessel or lumen, the method comprising:
positioning a combined imaging balloon catheter within the blood vessel or lumen, the combined imaging balloon catheter including:
a catheter having a distal end with a flexible tip having an outer diameter of 3 French or less;
an inflatable balloon assembly on the catheter proximal to the flexible tip; and
an imaging assembly in the flexible tip distal to the inflatable balloon assembly, the imaging assembly including an array of piezoelectric micromachined ultrasound transducer (PMUT) sensors;
imaging a portion of the blood vessel or lumen to be stented using the imaging assembly of the combined imaging balloon catheter without the inflatable balloon assembly being passed wholly through the portion of the blood vessel or wholly pulled back through the portion of the blood vessel or lumen, wherein imaging comprises applying a voltage between a plurality of electrode layers in each PMUT sensor of the array of PMUT sensors, wherein each PMUT sensor comprises a plurality of concentric multilayered stacks, each multilayered stack extending proud of a base layer over a cavity, further wherein each of the concentric multilayered stacks includes a plurality of piezoelectric layers, and wherein each piezoelectric layer is arranged between two electrode layers of the plurality of electrode layers; and inducing, from the applied voltage, a displacement that is a proportional to the applied voltage, a piezoelectric coefficient of a material forming the piezoelectric layers, and the number of piezoelectric layers; and
determining a stent size based at least in part on the imaging of the portion of the blood vessel or lumen.
